SEQUENCE LISTING (46 sequences)

SEQ ID NO: 1 - protein, 126 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 2 - protein, 107 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 3 - protein, 5 aa, synthetic construct
GYYMH

SEQ ID NO: 4 - protein, 17 aa, synthetic construct
WINPDSGGTNYAQKFQG

SEQ ID NO: 5 - protein, 17 aa, synthetic construct
DQPLGYCTNGVCSYFDY

SEQ ID NO: 6 - protein, 11 aa, synthetic construct
RASQGIYSWLA

SEQ ID NO: 7 - protein, 7 aa, synthetic construct
TASTLQS

SEQ ID NO: 8 - protein, 9 aa, synthetic construct
QQANIFPLT

SEQ ID NO: 9 - protein, 8 aa, synthetic construct
GYTFTGYY

SEQ ID NO: 10 - protein, 8 aa, synthetic construct
INPDSGGT

SEQ ID NO: 11 - protein, 19 aa, synthetic construct
ARDQPLGYCTNGVCSYFDY

SEQ ID NO: 12 - protein, 6 aa, synthetic construct
QGIYSW

SEQ ID NO: 13 - skipped

SEQ ID NO: 14 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTRFVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 117 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 16 - protein, 112 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 17 - protein, 8 aa, synthetic construct
GYSFTTYT

SEQ ID NO: 18 - protein, 8 aa, synthetic construct
INPSSGYA

SEQ ID NO: 19 - protein, 10 aa, synthetic construct
AREKFYGYDY

SEQ ID NO: 20 - protein, 11 aa, synthetic construct
QSIVHRNGNTY

SEQ ID NO: 21 - skipped

SEQ ID NO: 22 - protein, 9 aa, synthetic construct
FQGSHVPYT

SEQ ID NO: 23 - protein, 5 aa, synthetic construct
TYTIH

SEQ ID NO: 24 - protein, 17 aa, synthetic construct
YINPSSGYAAYNQKFQG

SEQ ID NO: 25 - protein, 8 aa, synthetic construct
EKFYGYDY

SEQ ID NO: 26 - protein, 16 aa, synthetic construct
KSSQSIVHRNGNTYLE

SEQ ID NO: 27 - protein, 7 aa, synthetic construct
KVSNRFS

SEQ ID NO: 28 - protein, 4 aa, synthetic construct
DYKD

SEQ ID NO: 29 - protein, 10 aa, synthetic construct
GDYKDDDDKG

SEQ ID NO: 30 - protein, 8 aa, synthetic construct
DYKDDDDK

SEQ ID NO: 31 - protein, 9 aa, synthetic construct
MDYKDDDDK

SEQ ID NO: 32 - protein, 7 aa, synthetic construct
DFKDDDK

SEQ ID NO: 33 - protein, 8 aa, synthetic construct
DYKAFDNL

SEQ ID NO: 34 - protein, 7 aa, synthetic construct
DYKDHDG

SEQ ID NO: 35 - protein, 9 aa, synthetic construct
MDFKDDDDK

SEQ ID NO: 36 - protein, 9 aa, synthetic construct
MDYKAFDNL

SEQ ID NO: 37 - protein, 7 aa, synthetic construct
DYKDHDI

SEQ ID NO: 38 - protein, 5 aa, synthetic construct
DYKDH

SEQ ID NO: 39 - protein, 5 aa, synthetic construct
DYKDD

SEQ ID NO: 40 - protein, 6 aa, synthetic construct
DYKDHD

SEQ ID NO: 41 - protein, 6 aa, synthetic construct
DYKDDD

SEQ ID NO: 42 - protein, 214 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 711 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 44 - DNA, 2133 bp, synthetic construct
  source 1..2133: mol_type = other DNA
caagtgcaactagtgcaaagtggtgcagaagtcaagaagcccggcgcttccgtgaaggtatcatgcaaggcctctggatacaccttcaccggctactacatgcattgggtcagacaggcccccggacaaggtctggaatggatgggctggatcaaccctgactccggcggaaccaactacgcccagaagtttcagggacgcgtgaccatgaccagagacacaagcatctccaccgcttatatggagctgaaccggctgagatccgacgacaccgccgtgtactactgtgctagagaccagcctctgggctactgcaccaatggcgtgtgctcctacttcgactactggggccagggaaccctggtgacagtgtcctctgccagcaccaaaggcccttctgtgttccccctggccccttgctctcggagcacctccgagtctaccgctgctctgggctgcctggtgaaggattactttcccgagcctgtgaccgtctcttggaactccggcgccctcacctccggcgtgcacacctttcctgctgtgctgcagtccagcggcctgtactctctgtcctctgtggtgaccgtgccttcttccaacttcggcacacagacctacacctgcaacgtggaccacaagcctagcaataccaaggtagataagacggtggaacggaagtgctgcgtggagtgccctccgtgtcctgcccctcctgtggcaggcccatctgtgtttctgttccctccaaagcccaaggacaccctgatgatctccagaacccctgaggtgacctgtgtggtggtcgatgtgtctcacgaggaccctgaagtgcagttcaactggtacgtggacggagtggaagtgcataacgccaagaccaaacctcgggaagagcagttcaacagcaccttcagagtggtgtccgtgctgaccgtggtacaccaggactggctgaacggcaaagagtacaagtgcaaggtgtctaacaagggcctgcctgctccaatcgagaagacgatttccaaaacaaagggccagcccagagaacctcaggtgtacacactgcctccatctagggaggaaatgaccaagaaccaggtgtccctgacctgtctggtcaagggcttctacccctccgacatcgccgtggaatgggagagcaatggacagcctgagaacaactacaaaaccacacctcctatgctggactctgacggctcattcttcctgtactccaagctgaccgtggataagtccagatggcagcagggcaatgtgttcagctgttctgtgatgcacgaggccctgcacaaccactacacccagaagtccctgtctctcagccctgggaagggcggaggaggctccggaggcggcggctctggaggtggaggcagcgatgtcgtgatgacccaatctcctgatagcctggctgtgtccttgggcgagagagccaccatcaactgcaagtcttctcagtctatcgtgcaccggaacggcaacacctacctcgagtggtaccagcagaaaccaggccaacctcctaagctgctgatctacaaggtgtccaacagattctctggcgtccccgaccggttctccggctccggctccggcaccgacttcaccctgaccatctcctccctgcaggctgaggacgtggccgtgtattattgcttccagggctcccacgtgccctacacctttggcggcggcaccaagctggagatcaagggtggcggcggctctggcggcggcggctccggaggtggtggctcccaagtgcagctggtgcagtccggcgccgaggtgaagaaacctggcgcttctgtgaaagtgtcctgcaaggcctctggctactccttcaccacctatacaatccactgggtgcggcaggcccctggccaaggcctggaatggatcggctacatcaacccctccagtggctacgccgcttacaaccagaagttccagggcagagtgaccctgaccgccgacaagtccacctctacagcctacatggaactgtcctctctgcggtctgaggataccgctgtgtactactgcgctagagagaagttctacggctacgactactggggccagggcaccaccgtgacagtctcctct

SEQ ID NO: 45 - DNA, 642 bp, synthetic construct
  source 1..642: mol_type = other DNA
gatatccaaatgactcaaagtccaagtagtgtgtccgcttccgtgggcgaccgcgtgaccatcacctgtagagcctcgcagggcatctattcttggctggcttggtaccagcagaagcctggcaaggctcctaatctgctgatctacaccgcctctaccctccagtccggcgtgccttcccggttctccggctctggaagcggcaccgacttcaccctgactatctcctctctgcagcccgaggactttgctacctactactgccagcaggccaacatcttccctctgaccttcggcggaggaaccaaagtggaaatcaagcggaccgtggccgcccctagcgtgttcatctttccaccttctgatgaacaactgaagtctggcaccgcttctgtggtgtgcctgctgaacaacttctaccctagagaggccaaagtgcagtggaaggtggacaacgccctgcagtctggcaactcccaagagtccgtcaccgagcaggattctaaggactccacctactccctgtctagcacactgacactgtccaaggccgactacgagaagcacaaggtgtacgcctgcgaagtgacccaccagggcctgagctcccccgtcacaaagtccttcaacagaggcgagtgc

SEQ ID NO: 46 - protein, 107 aa, synthetic construct [sequence omitted]

(19) United States
(12) Patent Application Publication
(10) Pub. No.: US 2026/0355315 A1
(43) Pub. Date: Nov. 12, 2026

(54) A METHOD OF RESCUING EXHAUSTED IMMUNE CELLS

(71) Applicant: Currus Biologics Pty Ltd, Melbourne (AU)

(72) Inventors: Michael Kershaw, Ocean Grove (AU); Clare Slaney, Greensborough, Victoria (AU); Bianca Von Scheidt, Ascot Vale, Victoria (AU)

(73) Assignee: Currus Biologics Pty Ltd, Melbourne (AU)

(21) Appl. No.: 19/112,536

(22) PCT Filed: Sep. 20, 2023
(86) PCT No.: PCT/AU2023/050904

§ 371 Date: Mar. 17, 2025

(30) Foreign Application Priority Data
Sep. 20, 2022 (AU) 2022902711

(51) Int. Cl. A61K 40/24; A61K 35/17; A61K 40/11; A61K 40/31; A61K 40/42; A61P 37/04; C07K 16/28; C12N 5/0783

(52) U.S. Cl. CPC A61K 40/24; A61K 35/17; A61K 40/11; A61K 40/31; A61K 40/4224; A61P 37/04; C07K 16/2878; C12N 5/0636; A61K 2239/13; A61K 2239/29; C07K 2317/31; C07K 2317/33; C07K 2317/565; C07K 2317/567; C12N 2510/00

(57) ABSTRACT

The invention is directed to compositions and methods for preventing or rescuing exhaustion of immune effector cells, especially CAR T cells. The compositions and methods comprise bispecific polypeptides with a first antigen binding protein that specifically binds to an antigen expressed on an antigen presenting cell (e.g. CD40) and a second antigen binding protein that specifically binds to an antigen on the immune effector cell (e.g. a FLAG tag on CAR T cells).

### FIELD OF THE INVENTION

The present disclosure relates generally to methods of reducing, preventing or rescuing exhausted immune cells expressing engineered receptors (such as chimeric antigen receptors or modified TCRs).

### RELATED APPLICATION

This application claims priority from Australian provisional application AU 2022902711, the entire contents of which are hereby incorporated by reference.

### BACKGROUND OF THE INVENTION

Immune cell exhaustion is an acquired state of immune cell dysfunction. Specifically, T cell exhaustion, which is an acquired state of T cell dysfunction, is a hallmark of cancer and chronic viral infection. Recently, treatments to reverse T cell exhaustion in cancer have proven strikingly effective.

The use of engineered immune cells expressing genetically engineered antigen receptors (such as chimeric antigen receptors (CAR) T cells) has also proven effective for the treatment of hematologic malignancies. However, the development of exhaustion in CAR T cells remains a significant barrier to the broader use of this technology, particularly in the treatment of solid tumours.

T cell exhaustion (including CAR T exhaustion) is characterized by a progressive decline in effector function, manifesting typically as a loss in T cell polyfunctionality. Exhausted T cells typically exhibit increased expression of inhibitory receptors (such as PD-1, LAG-3, and PD-L1), decreased production of effector cytokines, decreased proliferation rates, and decreased target cell killing activity. Revitalization of exhausted T cells can reinvigorate immunity.

Current strategies for modulating T cell exhaustion rely on directly modulating expression of effector gene expression products, such as immune checkpoints. Such modulation produces undesired side effects since physiological levels of such effector gene expression products are often required for normal T cell function. In addition, such strategies are vulnerable to drug resistance and can lead to immunopathology.

Accordingly, there is a need to develop new compositions and new methods for preventing or rescuing immune cell exhaustion, in particular T cell exhaustion.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method for rescuing immune effector cell activity or reversing immune effector cell exhaustion, the method comprising exposing an immune effector cell having or suspected of having an exhaustion phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cell,
optionally wherein the immune effector cell expresses a heterologous receptor comprising an antigen binding protein and a signalling protein, thereby rescuing immune effector cell activity or reversing immune effector cell exhaustion.

The present invention also provides a method for increasing immune effector cell activity or cytotoxicity of exhausted immune effector cells, the method comprising exposing a plurality of immune effector cells exhibiting one or more signs of an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cells,
optionally wherein the immune effector cells express heterologous receptors comprising an antigen binding protein and a signalling protein, thereby increasing immune cell effector activity or cytotoxicity.

The present invention also provides a method for preventing, inhibiting, reducing the progression of, or delaying the onset of, immune effector cell exhaustion, the method comprising exposing a plurality of immune effector cells having or suspected of having an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cells,
optionally wherein the immune effector cells express heterologous receptors comprising an antigen binding protein and a signalling protein,
thereby preventing, inhibiting, reducing the progression of, or delaying the onset of, immune effector cell exhaustion.

The present invention also provides a method for treating a disease or condition characterised by exhaustion of immune effector cells, the method comprising administering to a subject in need thereof, an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on immune effector cells of the subject,
thereby treating a disease or condition characterised by exhaustion of immune effector cells.

In any embodiment, the immune effector cell is selected from a T cell, an NK cell, an NKT cell or a γδ T cell. Preferably the effector immune cell is a T cell.

Preferably the heterologous receptor expressed by the immune effector cells is a chimeric antigen receptor (CAR) or an engineered T cell receptor (TCR) that binds to a target antigen (such as a tumour associated antigen). Accordingly it will be appreciated that in preferred embodiments, the immune effector cells are recombinant immune effector cells expressing a CAR or modified TCR, or immune effector cells derived therefrom.

Accordingly, in preferred embodiments, the immune effector cell is a T cell expressing a CAR (a CAR T cell). Optionally, the immune effector cell is a CAR NK cell or a CAR NKT cell.

In particularly preferred embodiments, there is provided a method for rescuing or reversing CAR T cell exhaustion, the method comprising exposing a plurality of CAR T cells, optionally wherein the cells have or are suspected of having an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the CAR T cells,
thereby rescuing or reversing CAR T cell exhaustion.

As used herein, rescuing or reversing immune cell effector exhaustion comprises reversing at least one marker or sign of an exhaustion phenotype in an immune effector cell.

Further, there is provided a method for increasing CAR T cell activity or cytotoxicity, the method comprising exposing a plurality of CAR T cells, preferably wherein the cells have or are suspected of having an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the CAR T cells,
thereby increasing CAR T cell activity or cytotoxicity.

Further, there is provided a method for preventing, inhibiting, reducing the progression of, or delaying the onset of, CAR T cell exhaustion, the method comprising exposing a plurality of CAR T cells, preferably wherein the cells have or are suspected of having an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the CAR T cells, thereby preventing, inhibiting, reducing or delaying the onset of CAR T exhaustion.

In any embodiment, exposing the immune effector cells (preferably CAR T cells) to the bispecific polypeptide comprises contacting a population of immune effector cells (eg CAR T cells) that have been obtained from a subject, with an effective amount of the bispecific polypeptide, ex vivo. Optionally, the method comprises the step of further administering the immune effector cells that have been contacted with the bispecific polypeptide, to the subject.

In any embodiment, exposing the immune cells (eg CAR T cells) to an effective amount of bispecific polypeptide is carried out in vivo. Accordingly, in such embodiments, the method comprises administration of the bispecific polypeptide to a subject that has received or is receiving a treatment with the immune effector cells (eg CAR T cells), such that the present invention provides a method of:
rescuing immune effector cell (eg CAR T cell) activity;
reversing immune effector cell (eg CAR T cell) exhaustion;
increasing immune effector cell (eg CAR T cell) activity or cytotoxicity; or
preventing, inhibiting, reducing progression of, or delaying the onset of, immune effector cell (eg CAR T cell) exhaustion;
in a subject, the method comprising administering to the subject, a bispecific polypeptide wherein the bispecific polypeptide comprises a first antigen binding protein that specifically binds to an antigen expressed on an antigen presenting cell (APC), preferably a professional APC, and a second binding antigen binding protein that specifically binds to an antigen on the immune (eg CAR T) cells.

In preferred embodiments, the bispecific polypeptide is administered to a subject, after the subject has received a treatment with CAR T cells (or other immune effector cell treatment). In such embodiments, preferably the bispecific polypeptide is administered to the subject at least 24 hrs, at least 48 hrs, at least 72 hrs, at least 96 hrs, at least 120 hrs, at least 144 hrs, at least 7 days, at least 8 days, at least 9 days, at least 10 days, at least 11 days, at least 12 days, at least 13 days, at least 1 week, at least 2 weeks, at least 3 weeks, at least 4 weeks, at least 5 weeks or at least 6 weeks or longer following administration of the CAR T cells (or other immune effector cell treatment) to the subject.

In any embodiment, at the time of administration of the bispecific polypeptide to the subject, an exhausted phenotype of one or more of the immune effector cells, or a marker or parameter indicative thereof, is detectable or measurable in the subject or in a biological sample from the subject.

In any embodiment, at the time of administration of the bispecific polypeptide to the subject, an exhausted phenotype of one or more of the immune effector cells, or a marker or parameter indicative thereof, has been detected or measured in the subject or in a biological sample from the subject.

Optionally, at least at or about 10%, at least at or about 20%, at least at or about 30%, at least at or about 40%, or at least at or about 50% of the total immune effector cells (preferably CAR T cells) in a biological sample from the subject has an exhaustion phenotype or at least one sign or marker of an exhaustion phenotype.

In any embodiment, the methods of the invention further comprise determining the risk or likelihood that a subject has immune effector cells (such as CAR T cells) with an exhaustion phenotype.

As such, the present invention also provides a method of treatment, the method comprising:
providing or identifying a subject who has received a treatment with CAR T cells;
determining that the subject likely has or is at risk of having CAR T cells with an exhausted phenotype;
administering to the subject, a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the CAR T cells.

Determining whether a subject has or is at risk of having immune effector cells (such as CAR T cells) with an exhausted phenotype may comprise determining the expression of one or more genes associated with immune cell exhaustion, in a sample of immune effector cells obtained from the subject.

In some embodiments, the exhaustion phenotype may comprise an increase in the level or degree of surface expression of a marker on the immune cells, or in the percentage of immune cells exhibiting surface expression, of one or more exhaustion markers, compared to a reference population of immune effector cells.

Examples of such genes or markers are described further herein and are known to the skilled person.

In any embodiment, the exhaustion phenotype, with reference to a T cell or population of T cells, comprises: an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions.

In some embodiments, the reference immune effector cell population may be: a population of immune effector cells known to have a non-exhausted phenotype; a population of naive T cells; a population of central memory T cells; or a population of stem central memory T cells. Optionally the cells are from the same subject, or of the same species as the subject, from which the immune effector cells having the exhausted phenotype are derived.

In some embodiments, the reference immune effector cell population (a) is a subject-matched population including bulk immune effector cells isolated from the blood of the subject from which the immune effector cells having the exhausted phenotype is derived, optionally wherein the bulk immune effector cells do not express the heterologous receptor (eg CAR) and/or (b) is obtained from the subject from which the immune effector cells having the exhausted phenotype is derived, prior to receiving administration of a dose of immune effector cells expressing the heterologous receptor (eg CAR). In other embodiments, the reference immune effector cell population is a composition including a sample of the immune effector cell therapy, or pharmaceutical composition including immune effector cells expressing the recombinant receptor, prior to its administration to the subject, optionally wherein the composition is a cryopreserved sample.

The sample may be any biological sample that comprises immune cells, such as a biological fluid, tissue or tumour. Preferably the biological sample is a sample of whole blood, or immune cells derived therefrom (such as a sample of peripheral blood mononuclear cells, PBMCs).

The present invention also provides a use of a bispecific polypeptide in the manufacture of a medicament for:
rescuing or reversing immune effector cell (eg CAR T cell) exhaustion;
increasing immune effector cell (eg CAR T cell) activity or cytotoxicity; or
preventing, inhibiting, reducing progression of, or delaying the onset of, immune effector cell (eg CAR T cell) exhaustion
in a subject, preferably wherein the subject has received or is receiving a treatment with an engineered immune effector cell (such as a CAR T cell).

The present invention also provides a bispecific polypeptide, or pharmaceutical composition comprising the same, for use in:
rescuing or reversing immune effector cell (eg CAR T cell) exhaustion;
increasing immune effector cell (eg CAR T cell) activity or cytotoxicity; or
preventing, inhibiting, reducing progression of, or delaying the onset of, immune effector cell (eg CAR T cell) exhaustion.

In any embodiment, the methods of the present invention are for rescuing exhaustion of immune effector cells in the context of any disease or condition selected from: cancer, a chronic infection (such as a chronic bacterial or chronic viral infection), chronic inflammation or autoimmunity. Accordingly, in certain embodiments, the methods of the present invention comprise methods for treating diseases or conditions such as cancer, chronic infection, chronic inflammation or autoimmunity, comprising administering to a subject in need thereof, a bispecific polypeptide as herein described.

As used herein, the bispecific polypeptide that comprises a first antigen binding protein that specifically binds to an antigen expressed on an antigen presenting cell (APC), preferably a professional APC, and a second binding antigen binding protein that specifically binds to an antigen on the CAR T cells, may otherwise be referred to herein as a “BEAT”.

Preferably the professional APC is an endogenous APC of the subject requiring treatment. Preferably the professional APC is selected from a dendritic cell, a macrophage and a B-cell, more preferably, a dendritic cell (DC). Accordingly, in preferred embodiments, the first antigen binding protein of the bispecific polypeptide can bind to any antigen of a professional APC, such as any selected from: MHCII, Clec9a, PD-L1, PD-L2, galectin, CD11c, CD19, CD40, CD206 and CD83.

Preferably the second antigen binding protein of the bispecific polypeptide specifically binds to an antigen on the CAR portion of the CAR T cell. The second antigen binding protein of the bispecific polypeptide may specifically bind to the antigen binding protein of the CAR, the hinge region of the CAR, a tag on the CAR (such as a Myc, Flag, His, HA, SBP, GST, MBP, GFP, S, Strep, exact or other tag) or any other region of the extracellular portion of the CAR.

Alternatively, the second antigen binding protein of the bispecific polypeptide may specifically bind to an antigen on the immune effector cell (optionally wherein the immune effector cell expresses a heterologous receptor such as a CAR). In such embodiments, the second antigen binding protein of the bispecific polypeptide may specifically bind to an antigen on the surface of the immune effector cell (eg T cell), such as: CD3, CD4, CD8, CD25, CD127, CD196 (CCR6), CD27, CD28, CD45RA, CD45RO, CD62L, CD197, and HLA-DR). In further embodiments, the immune effector cell may be engineered to express a heterologous antigen or tag in addition to the CAR receptor, and the second antigen binding protein of the bispecific polypeptide may bind to the heterologous antigen or tag.

In preferred embodiments, the bispecific polypeptide is in the form of a fusion protein, optionally wherein the first and second antigen binding proteins are directly linked or linked via a hinge or linker region. Suitable linkers are further described herein.

The first and second antigen binding proteins of the bispecific polypeptide are preferably in the form of an antibody, or antigen binding fragment thereof. In any embodiment, the first and/or second antigen binding proteins are in the form of an antibody, such as an immunoglobulin G (IgG1, IgG2, IgG3, or IgG4). Optionally, the first antigen binding proteins is in the form of an antibody, such as an immunoglobulin G (IgG1, IgG2, IgG3, or IgG4) and the second antigen binding protein is in the form of an antibody fragment (such as an scFv, a dimeric scFv (di-scFv), a Fab, a Fv, a F(ab′)2), wherein the second antigen binding protein is linked to the first antigen binding protein via the C or N terminus, or an internal region of the first antigen binding protein, preferably wherein the second antigen binding protein is linked to the first antigen binding protein via the C terminus of the heavy chain of the first antigen binding protein. Optionally, the second antigen binding proteins is in the form of an antibody, such as an immunoglobulin G (IgG1, IgG2, IgG3, or IgG4) and the first antigen binding protein is in the form of an antibody fragment (such as an scFv, a dimeric scFv (di-scFv), a Fab, a Fv, a F(ab′)2), wherein the first antigen binding protein is linked to the second antigen binding protein via the C or N terminus, or an internal region of the second antigen binding protein, preferably wherein the second antigen binding protein is linked to the second antigen binding protein via the C terminus of the heavy chain of the second antigen binding protein.

In any embodiment, the bispecific polypeptide comprises a first antigen binding protein for binding to CD40 and a second antigen binding protein for binding to a tag on a CAR of a CAR T cell.

In any embodiment, the bispecific polypeptide comprises a first antigen binding protein for binding to CD206 and a second antigen binding protein for binding to a tag on a CAR of a CAR T cell tag.

In any embodiment, the bispecific polypeptide comprises a first antigen binding protein for binding to CD40 and a second antigen binding protein for binding to a FLAG tag.

Preferably, the antigen binding protein for binding to CD40 is an activator of CD40.

In any embodiment, the bispecific polypeptide comprises a first antigen binding protein for binding to CD206 and a second antigen binding protein for binding to a FLAG tag.

In any embodiment, the bispecific polypeptide comprises a first antigen binding protein for binding to CD40, and wherein the first antigen binding protein comprises:
FR1-CDR1-FR2-CDR2-FR3-CDR3-FR4
and FR1a-CDR1a-FR2a-CDR2a-FR3a-CDR3a-FR4a,
wherein the sequence of any of the framework regions and/or complementarity determining regions are as described herein, preferably as described in Table 1.

In any embodiment, the first antigen binding protein competitively inhibits the binding to CD40 of an antibody or antigen binding fragment thereof, comprising a VH comprising a sequence as set forth in SEQ ID NO: 1 and a VL comprising a sequence as set forth in SEQ ID NO: 2.

In any embodiment the first antigen binding protein comprises a CDRH1, a CDRH2 and/or a CDRH3 of an antigen binding domain having a variable heavy domain as defined in SEQ ID NO: 1.

In any embodiment the first antigen binding protein comprises a CDRL1, a CDRL2 and/or a CDRL3 of an antigen binding domain having a variable light chain as defined in of SEQ ID NO: 2.

In any embodiment of the first antigen binding protein comprises a CDR1, a CDR2 and/or a CDR3 of an antigen binding domain having a variable heavy chain as defined in SEQ ID NO: 1 and a variable light chain as defined in SEQ ID NO: 2.

Preferably, the first antigen binding protein comprises:
FR1-CDR1-FR2-CDR2-FR3-CDR3-FR4-linker-FR1a-CDR1a-FR2a-CDR2a-FR3a-CDR3a-FR4a,
wherein the linker may be a chemical, one or more amino acids, or a disulphide bond formed between two cysteine residues.

Optionally, the first antigen binding protein comprises:
(i) a VH comprising a complementarity determining region (CDR) 1 comprising or consisting of a sequence as set forth in SEQ ID NO: 3, a CDR2 comprising or consisting of a sequence as set forth in SEQ ID NO: 4 and a CDR3 comprising or consisting of a sequence as set forth in SEQ ID NO: 5, and
(ii) a VL comprising a complementarity determining region (CDR) 1 comprising or consisting of a sequence as set forth in SEQ ID NO: 6, a CDR2 comprising or consisting of a sequence as set forth in SEQ ID NO: 7 and a CDR3 comprising or consisting of a sequence as set forth in SEQ ID NO: 8;
or
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 3, a CDR2 comprising or consisting of a sequence at least about 80%, at least 85%, at least 90%, at least 92%, at least 95%, at least 97%, at least 99% identical to a sequence set forth in SEQ ID NO: 4; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 5, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 6, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 7, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8.

In any embodiment the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

In any embodiment the first antigen binding protein comprises a light chain variable domain comprising or consisting of the amino acid sequence as set forth in SEQ ID NO: 2; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

In any embodiment, the second antigen binding protein is for binding to a FLAG tag, the second antigen binding protein comprising an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1.

In any embodiment, the second antigen binding protein is capable of specifically binding to a FLAG tag or variant thereof (such as defined in SEQ ID NOs: 28 or 30 or as otherwise defined herein). The antigen binding protein is preferably capable of specifically binding to protein domains comprising multiples of the FLAG tag sequences (eg 2×FLAG, 3×FLAG etc).

In any embodiment, the second antigen binding protein is for specifically binding to a peptide tag that contains or consists of the sequence DYK, preferably the sequence DYKD (SEQ ID NO: 28). In addition to these sequences, other amino acids can be present in the FLAG tag bound by the second antigen binding protein, preferably wherein the additional amino acids in the FLAG tag are hydrophilic amino acids for example R (Arg), D (Asp), E (Glu) and K (Lys) and/or amino acids with aromatic side chains for example Y (Tyr), F (Phe), H (His) and W (Trp).

In preferred embodiments the second antigen binding protein is capable of specifically binding to a FLAG-tag that contains, comprises or consists of the sequence GDYKDDDDKG (SEQ ID NO: 29), DYKDDDDK (SEQ ID NO: 30), MDYKDDDDK (SEQ ID NO: 31), DFKDDDK (SEQ ID NO: 32), DYKAFDNL (SEQ ID NO: 33), DYKDHDG (SEQ ID NO: 34), MDFKDDDDK (SEQ ID NO: 35), MDYKAFDNL (SEQ ID NO: 36), DYKDHDI (SEQ ID NO: 37), DYKDH (SEQ ID NO: 38), DYKDD (SEQ ID NO: 39), DYKDHD (SEQ ID NO: 40) and/or DYKDDD (SEQ ID NO: 41). The most preferred sequence is DYKDDDDK (SEQ ID NO: 30). The term FLAG-tag also encompasses modified FLAG-tags, such as those derived from the FLAG-tags described above, especially the tag with the sequence DYKDDDDK, by amino acid insertion, deletion or substitution.

In any embodiment, the FLAG tag is present at the N-terminus, the C-terminus or within the protein (eg a CAR) to which the bispecific polypeptide is capable of binding to, binds to or specifically binds to.

In any embodiment, the second antigen binding protein competitively inhibits the binding to a FLAG tag of an antibody comprising a VH comprising a sequence as set forth in SEQ ID NO: 15 and a VL comprising a sequence as set forth in SEQ ID NO: 16.

In any embodiment, the second antigen binding protein comprises a CDRH1, a CDRH2 and/or a CDRH3 of an antigen binding domain having a variable heavy chain as defined in SEQ ID NO: 15.

In any embodiment, the second antigen binding protein comprises a CDRL1, a CDRL2 and/or a CDRL3 of an antigen binding domain having a variable light chain as defined in SEQ ID NO: 16.

In any embodiment, the second antigen binding protein comprises a CDR1, a CDR2 and/or a CDR3 of an antigen binding domain having a variable heavy chain as defined in SEQ ID NO: 15 and a variable light chain as defined SEQ ID NO: 16.

In any embodiment the second antigen binding protein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1, and optionally, wherein the linker may be a chemical, one or more amino acids, or a disulphide bond formed between two cysteine residues.

In any embodiment, the second antigen binding domain comprises an antigen binding domain comprising:
a VH comprising a complementarity determining region CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 23; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 24; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 25, and
a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 26; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 27; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 22.

In any embodiment, the second antigen binding protein comprises an antigen binding domain comprising:
a VH comprising a CDR1 comprising or consisting of an amino acid sequence of SEQ ID NO: 23; a CDR2 comprising or consisting of an amino acid sequence of SEQ ID NO: 24; a CDR3 comprising or consisting of an amino acid sequence of SEQ ID NO: 25, and
a VL comprising a CDR1 comprising or consisting of an amino acid sequence of SEQ ID NO: 26; a CDR2 comprising or consisting of an amino acid sequence of SEQ ID NO: 27; a CDR3 comprising or consisting of an amino acid sequence of SEQ ID NO: 22.

In any embodiment, the second antigen binding protein comprises an antigen binding domain comprising a variable heavy chain comprising the amino acid sequence as set forth in SEQ ID NO: 15, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

In any embodiment, the second antigen binding protein comprises an antigen binding domain comprising a variable light chain comprising the amino acid sequence as set forth in SEQ ID NO: 16; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

In any embodiment of any aspect of the invention, the bispecific polypeptide comprises, consists essentially of or consists of an amino acid sequence of SEQ ID NOS: 42 and 43.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Schematic of the experimental protocol for stimulating CAR T cells with either highly expressing mesothelin positive tumour cells or BEAT and monocyte derived DCs (MoDCs) and restimulation with either tumour cells or MoDCs.

FIG. 2. Activation and exhaustion of CAR T cells 6 days after stimulating CAR T cells with either highly expressing mesothelin positive tumour cells or BEAT and monocyte derived DCs (MoDCs) and following restimulation with either tumour cells or MoDCs.

FIG. 3. CAR T cell phenotypes after stimulating CAR T cells with either highly expressing mesothelin positive tumour cells or BEAT and monocyte derived DCs (MoDCs) and following restimulation with either tumour cells or MoDCs.

FIG. 4. Schematic of the experimental protocol for sorting pre-stimulated CAR T cells and then re-culturing CAR T cells with mesothelin positive tumour cells, mesothelin negative tumour cells, monocyte derived DCs (MoDCs) or monocyte derived DCs (MoDCs) combined with BEAT for further stimulation and evaluation of proliferation and phenotype.

FIG. 5. T cell phenotype of BEAT and MoDCs (A) or tumour (B) pre-treated CAR T cells with either BEAT and MoDCs, negative tumour or positive tumour restimulation. BEAT and MoDC pre-treated CAR T cells have a higher central memory (TCM) and stem cell memory (TSCM) phenotype than tumour pre-treated CAR T cells which have a higher effector memory phenotype (TEM).

FIG. 6. T Cell proliferation when pre-treated CAR T cells with either tumour restimulation (A) or BEAT and MoDCs restimulation (B). BEAT and MoDC pre-treated CAR T cells have a higher proliferation than tumour pre-treated CARs.

### DESCRIPTION OF SEQUENCES

TABLE 1
Exemplary sequences

| | | |
|---|---|---|
| SEQ | | |
| ID NO | Descriptor | Sequence |

CD40 binding polypeptide

| | | |
|---|---|---|
| 1 | Anti-hCD40 VH | QVQLVQSGAEVKKPGASVKVSCKASGYTFTGYYMHWVRQAPGQGL |
| | | EWMGWINPDSGGTNYAQKFQGRVTMTRDTSISTAYMELNRLRSDD |
| | | TAVYYCARDQPLGYCTNGVCSYFDYWGQGTLVTVSS |
| 2 | Anti-hCD40 VL | DIQMTQSPSSVSASVGDRVTITCRASQGIYSWLAWYQQKPGKAPNL |
| | | LIYTASTLQSGVPSRFSGSGSGTDFTLTISSLQPEDFATYYCQQANIF |
| | | PLTFGGGTKVEIK |
| 3 | Anti-hCD40 | GYYMH |
| | CDRH1 (Kabat) | |
| 4 | Anti-hCD40 | WINPDSGGTNYAQKFQG |
| | CDRH2 (Kabat) | |
| 5 | Anti-hCD40 | DQPLGYCTNGVCSYFDY |
| | CDRH3 (Kabat) | |
| 6 | Anti-hCD40 CDRL1 | RASQGIYSWLA |
| | (Kabat) | |
| 7 | Anti-hCD40 CDRL2 | TASTLQS |
| | (Kabat) | |
| 8 | Anti-hCD40 CDRL3 | QQANIFPLT |
| | (Kabat, IMGT) | |
| 9 | Anti-hCD40 | GYTFTGYY |
| | CDRH1 (IMGT) | |
| 10 | Anti-hCD40 | INPDSGGT |
| | CDRH2 (IMGT) | |
| 11 | Anti-hCD40 | ARDQPLGYCTNGVCSYFDY |
| | CDRH3 (IMGT) | |
| 12 | Anti-hCD40 CDRL1 | QGIYSW |
| | (IMGT) | |
| 13 | Anti-hCD40 CDRL2 | TA |
| | (IMGT) | |
| 14 | anti-hCD40 IgG2 | ASTKGPSVFPLAPCSRSTSESTAALGCLVKDYFPEPVTVSWNSGALT |
| | CH | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCV |
| | | VVDVSHEDPEVQFNWYVDGVEVHNAKTKPREEQFNSTRFVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVMHEALHNHYTQKSLSLSPGK |
| 15 | anti-hCD40 IgG2 | RTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQG |
| | | LSSPVTKSFNRGEC |

FLAG binding polypeptides

| | | |
|---|---|---|
| 15 | anti-FLAG tag | QVQLVQSGAEVKKPGASVKVSCKASGYSFTTYTIHWVRQAPGQGLE |
| | hIgG1 VH | WIGYINPSSGYAAYNQKFQGRVTLTADKSTSTAYMELSSLRSEDTAV |
| | | YYCAREKFYGYDYWGQGTTVTVSS |
| 16 | anti-FLAG tag IgG1 | DVVMTQSPDSLAVSLGERATINCKSSQSIVHRNGNTYLEWYQQKPG |
| | VL | QPPKLLIYKVSNRFSGVPDRFSGSGSGTDFTLTISSLQAEDVAVYYCF |
| | | QGSHVPYTFGGGTKLEIK |
| 17 | Anti-FLAG CDRH1 | GYSFTTYT |
| | (IMGT) | |
| 18 | Anti-FLAG CDRH2 | INPSSGYA |
| | (IMGT) | |
| 19 | Anti-FLAG CDRH3 | AREKFYGYDY |
| | (IMGT) | |
| 20 | Anti-FLAG CDRL1 | QSIVHRNGNTY |
| | (IMGT) | |
| 21 | Anti-FLAG CDRL2 | KVS |
| | (IMGT) | |
| 22 | Anti-FLAG CDRL3 | FQGSHVPYT |
| | (IMGT, Kabat) | |
| 23 | Anti-FLAG CDRH1 | TYTIH |
| | (Kabat) | |
| 24 | Anti-FLAG CDRH2 | YINPSSGYAAYNQKFQG |
| | (Kabat) | |
| 25 | Anti-FLAG CDRH3 | EKFYGYDY |
| | (Kabat) | |
| 26 | Anti-FLAG CDRL1 | KSSQSIVHRNGNTYLE |
| | (Kabat) | |
| 27 | Anti-FLAG CDRL2 | KVSNRFS |
| | (Kabat) | |
| 28 | FLAG tag | DYKD |
| 29 | FLAG tag deca- | GDYKDDDDKG |
| | peptide | |
| 30 | FLAG tag | DYKDDDDK |
| | octapeptide | |
| 31 | FLAG tag | MDYKDDDDK |
| | nonapeptide | |
| 32 | FLAG tag | DFKDDDK |
| | heptapeptide | |
| 33 | FLAG tag | DYKAFDNL |
| | alternative | |
| | octapeptide | |
| 34 | FLAG tag | DYKDHDG |
| | alternative | |
| | heptapeptide | |
| 35 | FLAG tag | MDFKDDDDK |
| | alternative | |
| 36 | FLAG tag | MDYKAFDNL |
| | alternative | |
| 37 | FLAG tag | DYKDHDI |
| | alternative | |
| 38 | FLAG tag | DYKDH |
| | alternative | |
| 39 | FLAG tag | DYKDD |
| | alternative | |
| 40 | FLAG tag | DYKDHD |
| | alternative | |
| 41 | FLAG tag | DYKDDD |
| | alternative | |

Anti-CD40/anti-FLAG bispecific polypeptides

| | | |
|---|---|---|
| 42 | Anti-CD40 BEAT | DIQMTQSPSSVSASVGDRVTITCRASQGIYSWLAWYQQKPGKAPNL |
| | LC-IgG2 (anti- | LIYTASTLQSGVPSRFSGSGSGTDFTLTISSLQPEDFATYYCQQANIF |
| | CD40) | PLTFGGGTKVEIKRTVAAPSVFIFPPSDEQLKSGTASVVCLLNNFYPR |
| | | EAKVQWKVDNALQSGNSQESVTEQDSKDSTYSLSSTLTLSKADYEK |
| | | HKVYACEVTHQGLSSPVTKSFNRGEC |
| 43 | Anti-CD40-anti- | QVQLVQSGAEVKKPGASVKVSCKASGYTFTGYYMHWVRQAPGQGL |
| | FLAG BEAT | EWMGWINPDSGGTNYAQKFQGRVTMTRDTSISTAYMELNRLRSDD |
| | HC-IgG2(anti- | TAVYYCARDQPLGYCTNGVCSYFDYWGQGTLVTVSSASTKGPSVFP |
| | CD40)-(GGGGS)3- | LAPCSRSTSESTAALGCLVKDYFPEPVTVSWNSGALTSGVHTFPAVL |
| | anti-Flag -linker | QSSGLYSLSSVVTVPSSNFGTQTYTCNVDHKPSNTKVDKTVERKCC |
| | | VECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNG |
| | | KEYKCKVSNKGLPAPIEKTISKTKGQPREPQVYTLPPSREEMTKNQV |
| | | SLTCLVKGFYPSDIAVEWESNGQPENNYKTTPPMLDSDGSFFLYSKL |
| | | TVDKSRWQQGNVFSCSVMHEALHNHYTQKSLSLSPGKGGGGSGG |
| | | GGSGGGGSDVVMTQSPDSLAVSLGERATINCKSSQSIVHRNGNTYL |
| | | EWYQQKPGQPPKLLIYKVSNRFSGVPDRFSGSGSGTDFTLTISSLQA |
| | | EDVAVYYCFQGSHVPYTFGGGTKLEIKGGGGSGGGGSGGGGSQV |
| | | QLVQSGAEVKKPGASVKVSCKASGYSFTTYTIHWVRQAPGQGLEWI |
| | | GYINPSSGYAAYNQKFQGRVTLTADKSTSTAYMELSSLRSEDTAVYY |
| | | CAREKFYGYDYWGQGTTVTVSS |
| 44 | DNA | CAAGTGCAACTAGTGCAAAGTGGTGCAGAAGTCAAGAAGCCCGG |
| | Anti-CD40/anti- | CGCTTCCGTGAAGGTATCATGCAAGGCCTCTGGATACACCTTCAC |
| | FLAG BEAT HC | CGGCTACTACATGCATTGGGTCAGACAGGCCCCCGGACAAGGTC |
| | | TGGAATGGATGGGCTGGATCAACCCTGACTCCGGCGGAACCAAC |
| | | TACGCCCAGAAGTTTCAGGGACGCGTGACCATGACCAGAGACAC |
| | | AAGCATCTCCACCGCTTATATGGAGCTGAACCGGCTGAGATCCGA |
| | | CGACACCGCCGTGTACTACTGTGCTAGAGACCAGCCTCTGGGCT |
| | | ACTGCACCAATGGCGTGTGCTCCTACTTCGACTACTGGGGCCAG |
| | | GGAACCCTGGTGACAGTGTCCTCTGCCAGCACCAAAGGCCCTTC |
| | | TGTGTTCCCCCTGGCCCCTTGCTCTCGGAGCACCTCCGAGTCTAC |
| | | CGCTGCTCTGGGCTGCCTGGTGAAGGATTACTTTCCCGAGCCTG |
| | | TGACCGTCTCTTGGAACTCCGGCGCCCTCACCTCCGGCGTGCAC |
| | | ACCTTTCCTGCTGTGCTGCAGTCCAGCGGCCTGTACTCTCTGTCC |
| | | TCTGTGGTGACCGTGCCTTCTTCCAACTTCGGCACACAGACCTAC |
| | | ACCTGCAACGTGGACCACAAGCCTAGCAATACCAAGGTAGATAAG |
| | | ACGGTGGAACGGAAGTGCTGCGTGGAGTGCCCTCCGTGTCCTGC |
| | | CCCTCCTGTGGCAGGCCCATCTGTGTTTCTGTTCCCTCCAAAGCC |
| | | CAAGGACACCCTGATGATCTCCAGAACCCCTGAGGTGACCTGTGT |
| | | GGTGGTCGATGTGTCTCACGAGGACCCTGAAGTGCAGTTCAACT |
| | | GGTACGTGGACGGAGTGGAAGTGCATAACGCCAAGACCAAACCT |
| | | CGGGAAGAGCAGTTCAACAGCACCTTCAGAGTGGTGTCCGTGCT |
| | | GACCGTGGTACACCAGGACTGGCTGAACGGCAAAGAGTACAAGT |
| | | GCAAGGTGTCTAACAAGGGCCTGCCTGCTCCAATCGAGAAGACG |
| | | ATTTCCAAAACAAAGGGCCAGCCCAGAGAACCTCAGGTGTACACA |
| | | CTGCCTCCATCTAGGGAGGAAATGACCAAGAACCAGGTGTCCCT |
| | | GACCTGTCTGGTCAAGGGCTTCTACCCCTCCGACATCGCCGTGG |
| | | AATGGGAGAGCAATGGACAGCCTGAGAACAACTACAAAACCACAC |
| | | CTCCTATGCTGGACTCTGACGGCTCATTCTTCCTGTACTCCAAGC |
| | | TGACCGTGGATAAGTCCAGATGGCAGCAGGGCAATGTGTTCAGC |
| | | TGTTCTGTGATGCACGAGGCCCTGCACAACCACTACACCCAGAAG |
| | | TCCCTGTCTCTCAGCCCTGGGAAGGGCGGAGGAGGCTCCGGAG |
| | | GCGGCGGCTCTGGAGGTGGAGGCAGCGATGTCGTGATGACCCA |
| | | ATCTCCTGATAGCCTGGCTGTGTCCTTGGGCGAGAGAGCCACCA |
| | | TCAACTGCAAGTCTTCTCAGTCTATCGTGCACCGGAACGGCAACA |
| | | CCTACCTCGAGTGGTACCAGCAGAAACCAGGCCAACCTCCTAAG |
| | | CTGCTGATCTACAAGGTGTCCAACAGATTCTCTGGCGTCCCCGAC |
| | | CGGTTCTCCGGCTCCGGCTCCGGCACCGACTTCACCCTGACCAT |
| | | CTCCTCCCTGCAGGCTGAGGACGTGGCCGTGTATTATTGCTTCCA |
| | | GGGCTCCCACGTGCCCTACACCTTTGGCGGCGGCACCAAGCTGG |
| | | AGATCAAGGGTGGCGGCGGCTCTGGCGGCGGCGGCTCCGGAGG |
| | | TGGTGGCTCCCAAGTGCAGCTGGTGCAGTCCGGCGCCGAGGTG |
| | | AAGAAACCTGGCGCTTCTGTGAAAGTGTCCTGCAAGGCCTCTGG |
| | | CTACTCCTTCACCACCTATACAATCCACTGGGTGCGGCAGGCCCC |
| | | TGGCCAAGGCCTGGAATGGATCGGCTACATCAACCCCTCCAGTG |
| | | GCTACGCCGCTTACAACCAGAAGTTCCAGGGCAGAGTGACCCTG |
| | | ACCGCCGACAAGTCCACCTCTACAGCCTACATGGAACTGTCCTCT |
| | | CTGCGGTCTGAGGATACCGCTGTGTACTACTGCGCTAGAGAGAA |
| | | GTTCTACGGCTACGACTACTGGGGCCAGGGCACCACCGTGACAG |
| | | TCTCCTCT |
| 45 | DNA | GATATCCAAATGACTCAAAGTCCAAGTAGTGTGTCCGCTTCCGTG |
| | Anti-CD40/anti- | GGCGACCGCGTGACCATCACCTGTAGAGCCTCGCAGGGCATCTA |
| | FLAG BEAT LC | TTCTTGGCTGGCTTGGTACCAGCAGAAGCCTGGCAAGGCTCCTAA |
| | | TCTGCTGATCTACACCGCCTCTACCCTCCAGTCCGGCGTGCCTTC |
| | | CCGGTTCTCCGGCTCTGGAAGCGGCACCGACTTCACCCTGACTA |
| | | TCTCCTCTCTGCAGCCCGAGGACTTTGCTACCTACTACTGCCAGC |
| | | AGGCCAACATCTTCCCTCTGACCTTCGGCGGAGGAACCAAAGTG |
| | | GAAATCAAGCGGACCGTGGCCGCCCCTAGCGTGTTCATCTTTCCA |
| | | CCTTCTGATGAACAACTGAAGTCTGGCACCGCTTCTGTGGTGTGC |
| | | CTGCTGAACAACTTCTACCCTAGAGAGGCCAAAGTGCAGTGGAAG |
| | | GTGGACAACGCCCTGCAGTCTGGCAACTCCCAAGAGTCCGTCAC |
| | | CGAGCAGGATTCTAAGGACTCCACCTACTCCCTGTCTAGCACACT |
| | | GACACTGTCCAAGGCCGACTACGAGAAGCACAAGGTGTACGCCT |
| | | GCGAAGTGACCCACCAGGGCCTGAGCTCCCCCGTCACAAAGTCC |
| | | TTCAACAGAGGCGAGTGC |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all the steps, features, compositions and compounds referred to or indicated in this specification, individually or collectively, and any and all combinations or any two or more of said steps or features.

All of the patents and publications referred to herein are incorporated by reference in their entirety.

Unless otherwise indicated, the recombinant protein, cell culture, and immunological techniques utilised in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The inventors have surprisingly shown that a bispecific polypeptide as described herein, for binding to a professional APC, and to an immune effector cell (such as a CAR T cell) rescues an exhaustion phenotype and promotes the activation and expansion of exhausted immune cells in vivo. Thus the methods of the invention relate, in part, to methods for improving the efficacy of immune cell therapies, such as CAR T cell therapies.

### DEFINITIONS

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. The protein may include one or more non-natural amino acids.

The term “polypeptide” or “polypeptide chain” will be understood from the foregoing paragraph to mean a series of contiguous amino acids linked by peptide bonds.

Amino acids may be referred to herein by their commonly used full name (e.g., cysteine), their commonly known three letter symbols (e.g., Cys), or by the one-letter symbols recommended by the IUPAC-IUB Biochemical Nomenclature Commission (e.g., C). Nucleotides, likewise, may be referred to by their commonly accepted single-letter codes.

The term “antibody” as used herein broadly refers to any immunoglobulin (lg) molecule comprised of four polypeptide chains, two heavy (H) chains and two light (L) chains. Also disclosed herein are antigen-binding fragments, mutants, variants, and derivatives thereof, which retain the essential epitope binding features of the antibody molecule. Such mutants, variants, and derivatives will be known to persons skilled in the art, illustrative examples of which are described elsewhere herein.

An antibody heavy chain will typically comprise a heavy chain variable region (HCVR or VH) and a heavy chain constant region. The heavy chain constant region is typically comprised of three domains, CH1, CH2 and CH3. A light chain will typically comprise a light chain variable region (LCVR or VL) and a light chain constant region, CL. The VH and VL regions can be further subdivided into regions of hypervariability, also known as complementary determining regions (CDR), interspersed with framework regions (FR). Each VH and VL is typically comprised of three CDs and four FR, arranged from amino-terminus to carboxy-terminus in the following order: FR1, CDR1, FR2, CDR2, FR3, CDR3 and FR4. Immunoglobulin molecules can be of any type (e.g. IgG, IgE, IgM, IgD, IgA and IgY), class (e.g. IgG1, IgG2, IgG3, IgG4, IgA1 and IgA2) or subclass.

The term “antigen-binding fragment” or “antibody fragment”, as used herein, means one or more fragments of an antibody that retain the ability to specifically bind to the target antigen. Illustrative examples of antigen-binding fragments include (i) a Fab fragment, a monovalent fragment consisting of the VL, VH, CL and CH1 domains; (ii) a F (ab′)2 fragment, a F(ab′)2 fragment, a bivalent fragment comprising two Fab fragments linked by a disulfide bridge at the hinge region; (iii) a Fd fragment consisting of the VH and CH1 domains; (iv) a single-chain variable fragment (scFv) consisting of the VL and VH domains of a single arm of an antibody, (v) a dAb fragment (Ward et al. 1989, Nature, 341:544-6), which comprises a single variable domain; and (vi) an isolated CDR.

As used herein, “variable region” refers to the portions of the light and/or heavy chains of an antibody as defined herein that is capable of specifically binding to an antigen and, includes amino acid sequences of complementarity determining regions (CDRs); i.e., CDR₁, CDR₂, and CDR₃, and framework regions (FRs). For example, the variable region comprises three or four FRs (e.g., FR₁, FR₂, FR₃ and optionally FR₄) together with three CDRs. V_{H} refers to the variable region of the heavy chain. V_{L} refers to the variable region of the light chain.

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989, Martin (“enhanced Chothia”; Mol Immunol. (2008) 45:3832-9; or Al-Lazikani et al., J Mol Biol 273, 927-948, 1997, or the IMGT system discussed in Giudicelli et al., Nucleic Acids Res. 25:206-211 1997.

“Framework regions” (FRs) are those variable region residues other than the CDR residues. The FRs of VH are also referred to herein as FR H₁, FR H₂, FR H₃ and FR H₄, respectively, wherein FR H₁ corresponds to FR 1 of V_{H}, FR H₂ corresponds to FR 2 of V_{H}, FR H₃ corresponds to FR 3 of V_{H} and FR H₄ corresponds to FR 4 of V_{H}. Likewise, the FRs of V_{L} are referred to herein as FR L₁, FR L₂, FR L₃ and FR L₄, respectively, wherein FR L₁ corresponds to FR 1 of V_{L}, FR L₂ corresponds to FR 2 of V_{L}, FR L₃ corresponds to FR 3 of V_{L} and FR L₄ corresponds to FR 4 of V_{L}.

As used herein, the term “Fv” shall be taken to mean any protein, whether comprised of multiple polypeptides or a single polypeptide, in which a V_{L} and a V_{H} associate and form a complex having an antigen binding domain, i.e., capable of specifically binding to an antigen. The V_{H} and the V_{L} that form the antigen binding domain can be in a single polypeptide chain or in different polypeptide chains. Furthermore, an Fv of the invention (as well as any protein of the invention) may have multiple antigen binding domains that may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH₂ or CH₃ domain, e.g., a minibody.

A “Fab fragment” consists of a monovalent antigen-binding fragment of an immunoglobulin and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH₃ domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “antigen” as used herein refers to a molecule bound by an “antibody”, “antibody fragment” or a “bispecific polypeptide”. Antigens may be proteins recognized by immunoglobulins, in which case the sites on the proteins bound by the immunoglobulins are referred to as “epitopes”.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to an antigen with materially greater affinity (e.g., 1.5 fold or 2 fold or 5 fold or 10 fold or 20 fold or 40 fold or 60 fold or 80 fold to 100 fold or 150 fold or 200 fold) than it does to other related molecules. In example of the present invention, an antigen binding protein that “specifically binds” to an antigen with an affinity at least 1.5 fold or 2 fold or greater (e.g., 5 fold or 10 fold or 20 fold r 50 fold or 100 fold or 200 fold) than it does to any other antigen. Generally, but not necessarily, reference to binding means specific binding, and each term shall be understood to provide explicit support for the other term.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

The terms “Chimeric Antigen Receptor” or “CAR” as used herein refer to a recombinant polypeptide comprising an antigen binding domain that is linked to at least one intracellular signaling domain. The antigen binding domain of a CAR is a functional portion of the CAR that specifically binds to (i.e., specifically targets) an antigen expressed on a cancer cell (i.e., a “tumour-associated antigen”). Examples of tumour-associated antigens are known to persons skilled in the art, illustrative examples of which include Her2, CEA, FBP, CD19 and BCMA.

As used herein “tumour-associated antigen” refers to an antigen that is expressed by cancer cells. A tumour-associated antigen may or may not be expressed by non-tumour cells. When a tumour-associated antigen is not expressed by non-tumour cells (i.e., when it is unique to tumour cells) it may also be referred to as a “tumour-specific antigen.” When a tumour-associated antigen is not unique to a tumour cell, it is also expressed on a non-tumour cell under conditions that fail to induce a state of immunologic tolerance to the antigen. The expression of the antigen on the tumour may occur under conditions that enable the immune system to respond to the antigen. Tumour-associated antigens may be antigens that are expressed on non-tumour cells during fetal development when the immune system is immature and unable to respond, or they may be antigens that are normally present at low levels on normal cells but which are expressed at much higher levels on tumour cells. Those tumour-associated antigens of greatest clinical interest are differentially expressed compared to the corresponding non-tumour tissue and allow for a preferential recognition of tumour cells by specific T-cells or immunoglobulins.

The term “cancer” as used herein means any condition associated with aberrant cell proliferation. Such conditions will be known to persons skilled in the art. In an embodiment, the cancer is a solid cancer. In another embodiment, the cancer is a Her2 positive cancer. In another embodiment, the cancer is selected from the group consisting of breast cancer, pancreatic cancer, and lung cancer.

The terms “treat”, “treatment” and “treating” as used herein refers to any and all uses which remedy a condition or symptom, or otherwise prevent, hinder, retard, abrogate or reverse the onset or progression of cancer or other undesirable symptoms in any way whatsoever. Thus, the term “treating” and the like are to be considered in their broadest possible context. For example, treatment does not necessarily imply that a subject is treated until total recovery or cure. In conditions that display or are characterized by multiple symptoms, the treatment need not necessarily remedy, prevent, hinder, retard, abrogate or reverse all of said symptoms, but may remedy, prevent, hinder, retard, abrogate or reverse one or more of said symptoms.

The terms “immune cell” or “immune effector cell” refer to a cell that may be part of the immune system, either the adaptive (i.e. cellular or humoral) or innate immune system, and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). The immune cell may be an artificial cell subset including induced pluripotent stem cells and cells maturated therefrom. Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

### Methods of Treatment

The present invention relates to methods for rescuing or reversing immune effector cell exhaustion, increasing immune effector cell activity or potency (particularly in the case of exhaustion), and preventing, inhibiting, reducing the progression of, or delaying the onset of, immune effector cell exhaustion.

As described elsewhere herein, the immune effector cells for which exhaustion is to be prevented or rescued, may be any immune effector cell, illustrative examples of which include T cells, tumour infiltrating lymphocytes (TIL) natural killer (NK) cells, natural killer T cells (NKT cells) and gamma-delta cells (γδ cells). The T cell may be a cytotoxic T cell (CTL; CD8+ T cell), CD4+cos+ T cell, CD4−cos− T cell, or any other subset of T cells. Other illustrative examples of suitable T cells include T cells expressing one or more of the following markers: CD3, CD4, CD8, CD27, CD2S, CD45RA, CD45RO, CD62L, CD127, CD197, and HLA-DR.

In preferred embodiments, the immune cell is one that expresses a heterologous chimeric antigen receptor (CAR) or modified T cell receptor (TCR). Such receptors and the use thereof in treating various conditions and diseases, especially cancers, are well known to the skilled person.

Sources of cells for use in accordance with the methods of the present invention will be known to persons skilled in the art, illustrative examples of which include peripheral blood, peripheral blood mononuclear cells, bone marrow, lymph nodes tissue, cord blood, thymus tissue, tissue from the site of infection, ascites, pleural effusion, spleen tissue, and tumours. In an embodiment, the cells are derived from whole blood.

In any embodiment, the immune cell is derived from an autologous cell. In another embodiment, the immune cell is derived from an allogeneic cell.

The term “autologous” refers to any material derived from the same individual to whom the material is later to be re-introduced to the individual.

The term “allogeneic” refers to any material derived from a different individual of the same species as the individual to whom the material is introduced. Two or more individuals are said to be allogeneic to one another when the genes at one or more loci are not identical. In some aspects, allogeneic materials from individuals of the same species may be sufficiently genetically distinct to interact antigenically.

It will be appreciated that in preferred embodiments of the invention, the methods relate to rescuing exhaustion of CAR T cells that have been administered to a subject and which require re-activating.

Administration of an immune cell expressing a CAR and a bispecific polypeptide or pharmaceutical composition as described herein may be achieved by formulating the immune cell and the bispecific polypeptide or pharmaceutical composition in the same composition (e.g., for simultaneous co-administration) or they may be formulated as different compositions for sequential administration.

It will be appreciated that given the focus of the methods of the invention to rescue immune cell exhaustion, to activate immune cells displaying an exhaustion phenotype, or to prevent, or delay immune cell exhaustion, sequential administration may be preferred.

By “sequential” administration is meant there is an interval between the administration of the immune cell and the bispecific polypeptide or pharmaceutical composition comprising the same. The interval between sequential administrations may be seconds, minutes, hours or days. Preferably, there is a period of at least 24 hrs, at least 48 hrs, at least 72 hrs, at least 96 hrs, at least 120 hrs, at least 144 hrs, at least 7 days, at least 8 days, at least 9 days, at least 10 days, at least 11 days, at least 12 days, at least 13 days, at least 1 week, at least 2 weeks, at least 3 weeks, at least 4 weeks, at least 5 weeks, at least 6 weeks or more, between the initial administration of the immune cell (eg CAR T cell) and the bispecific polypeptide.

In an embodiment, periodic re-administration of the bispecific polypeptide may be required to achieve a desired therapeutic effect.

In some embodiments, the administration of the bispecific polypeptide is carried out in a cycling regimen involving administration of an effective amount of the bispecific polypeptide (i) daily for a period of more than one week, (ii) per day for no more than 6 days per week for a period of more than one week, (ii) per day for no more than 5 days per week for a period of more than one week; or per day for no more than 4 days per week for a period of more than one week. In certain embodiments, the administration of the bispecific polypeptide is carried out in a cycling regimen involving administration of an effective amount of the bispecific polypeptide per day for no more than 5 days per week for a period of more than one week.

In some embodiments of any of the methods provided herein, the administration of the bispecific polypeptide is initiated at or after peak or maximum level of the cells of the T cell therapy are detectable in the blood of the subject. In some embodiments, the administration of the bispecific polypeptide is initiated about 14 to about 35 days after initiation of administration of the T cell therapy. In certain embodiments, the administration of the bispecific polypeptide is initiated about 21 to about 35 days after initiation of administration of the T cell therapy. In other embodiments, the administration of the bispecific polypeptide is initiated about 21 to about 28 days after initiation of administration of the T cell therapy. In some embodiments, the administration of the bispecific polypeptide is initiated at or about 21 days, at or about 22 days, at or about 23 days, at or about 24 days, at or about 25 days, at or about 26 days, at or about 27 days, or at or about 28 days after initiation of administration of the T cell therapy. In certain embodiments, the administration of the bispecific polypeptide is initiated at or about 28 days after the initiation of the administration of the T cell therapy. In some embodiments of any of the methods provided herein, at the time of the initiation of the administration of the bispecific polypeptide, the subject does not exhibit a severe toxicity following the administration of the T cell therapy.

In some embodiments of any of the methods provided herein, the administration of the bispecific polypeptide is initiated at or about 28 days after the initiation of the administration of the T cell therapy. In some embodiments of any of the methods provided herein, at the time of the initiation of the administration of the bispecific polypeptide, the subject does not exhibit a severe toxicity following the administration of the T cell therapy. In some embodiments, the severe toxicity is severe cytokine release syndrome (CRS), optionally grade 3 or higher, prolonged grade 3 or higher or grade 4 or 5 CRS; and/or the severe toxicity is severe neurotoxicity, optionally grade 3 or higher, prolonged grade 3 or higher or grade 4 or 5 neurotoxicity.

In some embodiments of any of the methods provided herein, the administration of the bispecific polypeptide is suspended and/or the cycling regimen is modified if the subject exhibits a toxicity following the administration of the bispecific polypeptide, optionally a hematologic toxicity. In some embodiments, the toxicity is selected from severe neutropenia, optionally febrile neutropenia, prolonged grade 3 or higher neutropenia. In some embodiments, the administration of the bispecific polypeptide is restarted after the subject no longer exhibits the toxicity. In some embodiments, the cycling regimen is modified after the administration of the bispecific polypeptide is restarted. In some embodiments, the modified cycling regimen involves administering a reduced amount of the bispecific polypeptide and/or decreasing frequency of the administration of the bispecific polypeptide. In certain embodiments, the modified cycling regimen involves administering a reduced amount of the bispecific polypeptide.

The methods of the present invention also contemplate ex vivo contacting of the immune cells with the bispecific polypeptide. For example, in a scenario where a patient received a treatment with immune cells (eg CAR T cells), and those cells are now displaying an exhaustion phenotype, the cells may be removed from the patient, and contact with the bispecific polypeptide, ex vivo, in order to rescue the exhaustion phenotype and reactive the immune cells. In such embodiments, the immune cells may also be subjected to a combination treatment with the bispecific polypeptide described herein and with another known immune cell activator. Examples of such immune cell activation include providing a primary stimulation signal through, for example, the T cell TCR/CD3 complex or via stimulation of the CD2 surface protein and by providing a secondary co-stimulation signal through an accessory molecule, e.g, CD28 or 4-IBBL. In addition to the primary stimulation signal provided through the TCR/CD3 complex, or via CD2, induction of T cell responses requires a second, costimulatory signal. In particular embodiments, a CD28 binding agent can be used to provide a costimulatory signal. Suitable costimulatory ligands include, but are not limited to, CD7, B7-1 (CD80), B7-2 (CD86), 4-IBBL, OX40L, inducible costimulatory ligand (ICOS-L), intercellular adhesion molecule (ICAM), CD30L, CD40, CD70, CD83, HLA-G, MICA, MICE, HVEM, lymphotoxin beta receptor, ILT3, ILT4, an agonist or antibody that binds Toll ligand receptor, and a ligand that specifically binds with B7-H3.

The subject requiring treatment may be a human or a mammal of economic importance and/or social importance to humans, for instance, carnivores other than humans (e.g., cats and dogs), swine (e.g., pigs, hogs, and wild boars), ruminants (e.g., cattle, oxen, sheep, giraffes, deer, goats, bison, and camels), horses, and birds including those kinds of birds that are endangered, kept in zoos, and fowl, and more particularly domesticated fowl, e.g., poultry, such as turkeys, chickens, ducks, geese, guinea fowl, and the like, as they are of economic importance to humans. The term “subject” does not denote a particular age. Thus, adult, juvenile and newborn subjects are intended to be covered.

The terms “subject”, “individual” and “patient” are used interchangeably herein to refer to any subject to which the present disclosure may be applicable. In an embodiment, the subject is a mammal. In another embodiment, the subject is a human.

The term “therapeutically effective amount” as used herein means the amount of bispecific polypeptide when administered to a mammal, in particular a human, in need of such treatment, is sufficient to rescue or prevent an exhaustion phenotype in immune effector cells (such as CAR T cells). The precise amount of bispecific polypeptide to be administered can be determined by a physician with consideration of individual differences in age, weight, tumor size, extent of infection or metastasis, and condition of the subject.

In any embodiment, the provided methods can potentiate T cell therapy, e.g. CAR-T cell therapy, which, in some aspects, can improve outcomes for treatment. In some embodiments, the methods are particularly advantageous in subjects in which the cells of the T cell therapy exhibit weak expansion, have become exhausted, exhibit a reduced or decreased persistence in the subject and/or in subjects that have a cancer that is resistant or refractory to other therapies, is an aggressive or high-risk cancer, and/or that is or is likely to exhibit a relatively lower response rate to a CAR-T cell therapy administered without the bispecific polypeptide compared to another type of cancer or compared to administration with a different CAR-T cell therapy.

In some embodiments, the provided methods are used at a time at which a T cell therapy (e.g. CAR T cells) may exhibit or are likely to exhibit features of exhaustion. In some embodiments, an exhaustive phenotype is evident after T cells, having reached peak expansion, begin to decline in number in the blood of the subject. In some embodiments, the methods of exposing or contacting T cells of a T cell therapy (CAR T cells) with a bispecific polypeptide as described herein, are carried out at a time at which the T cells exhibit an increase in a hypofunctional or exhaustive state compared to at the time just prior to exposure of the T cells to an antigen (baseline) or to a time point at which the cells have been exposed to the antigen but are continuing to proliferate and have not yet reached peak expansion. In some embodiments, an increase in hypofunctional or exhaustive state can be determined by increased expression of an exhaustion marker compared to the previous earlier time point. In some embodiments, the increase in the hypofunctional or exhaustive state, such as increase in expression of an exhaustion marker, is at a time following administration of the T cell therapy (e . . . g. CAR T cells) to a subject having a disease or condition associated with the antigen targeted by the T cell therapy. The T cells, such as T cells in peripheral blood after administration to a subject, can be monitored for markers of T cell activation or exhaustion such as PD-1, TIM-3 and LAG-3 or as further described herein.

Methods for determining exhaustion phenotypes of immune effector cells are known to the skilled person and include determining the expression of various genes by the immune cells, or determining the presence or absence of particular cell surface markers. In other words, the skilled person will be familiar with exhaustion markers and methods for assessing/determining same in a sample of immune cells.

In some embodiments of any of the methods provided herein, the exhaustion phenotype, with reference to a T cell or population of T cells, includes: an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions. In some embodiments, the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more. In other embodiments, the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

In some embodiments of any of the methods provided herein, the reference T cell population is a population of T cells known to have a non-exhausted phenotype, is a population of naive T cells, is a population of central memory T cells, or is a population of stem central memory T cells, optionally from the same subject, or of the same species as the subject, from which the T cell or T cells having the exhausted phenotype are derived.

In some embodiments of any of the methods provided herein, the reference T cell population (a) is a subject-matched population including bulk T cells isolated from the blood of the subject from which the T cell or T cells having the exhausted phenotype is derived, optionally wherein the bulk T cells do not express the recombinant receptor and/or (b) is obtained from the subject from which the T cell or T cells having the exhausted phenotype is derived, prior to receiving administration of a dose of T cells expressing the recombinant receptor. In some embodiments, the reference T cell population is a composition including a sample of the T cell therapy, or pharmaceutical composition including T cells expressing the recombinant receptor, prior to its administration to the subject, optionally wherein the composition is a cryopreserved sample.

In some embodiments of any of the methods provided herein, the one or more exhaustion marker is an inhibitory receptor. In some embodiments, the one or more exhaustion marker is selected from among PD-I, CTLA-4, TIM-3, LAG-3, BTLA, 2B4, CD160, CD39, VISTA, and TIGIT.

In some embodiments of any of the methods provided herein, the activity or is one or more of proliferation, cytotoxicity or production of one or a combination of inflammatory cytokines, optionally wherein the one or a combination of cytokines is selected from IL-2, IFN-gamma and TNF-alpha. Other markers of immune cell activation include CD25 and CD69. In some embodiments, the exposure to said antigen or antigen receptor-specific agent includes incubation with the antigen or antigen receptor-specific agent, optionally an agent that binds the recombinant receptor, wherein said antigen is optionally the target antigen. In some embodiments, the antigen or antigen receptor-specific agent includes antigen-expressing target cells, optionally cells of said disease, disorder or condition. In certain embodiments, the target antigen is a human antigen.

In accordance with the methods of the present invention, it is also within the purview of the skilled person to be able to assess the efficacy of treatment as described herein. In other words, the skilled person can readily determine whether immune cell exhaustion has been prevented, rescued or otherwise by assessing one or more markers of exhaustion or assessing one or more markers of immune cell activation.

In some embodiments of any of the methods provided herein, the administration of the bispecific polypeptide: reverses an exhaustion phenotype in recombinant receptor-expressing T cells in the subject; prevents, inhibits or delays the onset of an exhaustion phenotype in recombinant receptor-expressing T cells in the subject; or reduces the level or degree of an exhaustion phenotype in recombinant receptor-expressing T cells in the subject; or reduces the percentage, of the total number of recombinant receptor-expressing T cells in the subject, that have an exhaustion phenotype.

In some embodiments of any of the methods provided herein, the initiation of the administration of the bispecific polypeptide is carried out subsequently to the administration of the T cell therapy and, following administration of the bispecific polypeptide or initiation thereof, the subject exhibits a restoration or rescue of an antigen- or tumor-specific activity or function of recombinant receptor-expressing T cells in said subject, optionally wherein said restoration, rescue, and/or initiation of administration of said bispecific polypeptide, is at a point in time after recombinant receptor expressing T cells in the subject or the in the blood of the subject have exhibited an exhausted phenotype.

In some embodiments of any of the methods provided herein, the administration of the bispecific polypeptide includes administration at an amount, frequency and/or duration effective to: (a) effect an increase in antigen-specific or antigen receptor-driven activity of naive or non-exhausted T cells in the subject, which optionally include T cells expressing said recombinant receptor, following exposure of the T cells to antigen or to an antigen receptor-specific agent as compared to the absence of said administration of said bispecific polypeptide; or (b) prevent, inhibit or delay the onset of an exhaustion phenotype, in naive or non-exhausted T cells T cells in the subject, which optionally include T cells expressing said recombinant receptor, following exposure of the T cells to antigen or to an antigen receptor-specific agent, as compared to the absence of said administration of said bispecific polypeptide; or (c) reverse an exhaustion phenotype in exhausted T cells, optionally including T cells expressing said recombinant receptor, in the subject, as compared to the absence of said administration of said subject.

In some embodiments, the administration of the bispecific polypeptide includes administration at an amount, frequency and/or duration effective (i) to effect said increase in activity and (ii) to prevent, inhibit or delay said onset of said exhaustion phenotype and/or reverse said exhaustion phenotype. In some embodiments, the T cells in the subject include T cells expressing said recombinant receptor and/or said antigen is the target antigen.

In some embodiments of any of the methods provided herein, the dose of genetically engineered T cells for administration, either simultaneously or sequentially with respect to the bispecific polypeptide, includes from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5 ×10⁷ to 1×10⁸ total CAR-expressing T cells, each inclusive. In some embodiments of any of the methods provided herein, the dose of genetically engineered T cells includes at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In certain embodiments, the dose of genetically engineered T cells includes at or about 5×10⁷ total CAR-expressing T cells. In other embodiments, the dose of genetically engineered T cells includes at or about 1×10⁸ CAR-expressing cells.

In some embodiments of any of the methods provided herein, the dose of cells is administered parenterally, optionally intravenously.

In some embodiments of any of the methods provided herein, the T cells are primary T cells obtained from a subject. In some embodiments, the T cells are autologous to the subject. In other embodiments, the T cells are allogeneic to the subject.

In some embodiments of any of the methods provided herein, the dose of genetically engineered T cells includes CD4+ T cells expressing the CAR and CD8+ T cells expressing the CAR and the administration of the dose includes administering a plurality of separate compositions, said plurality of separate compositions including a first composition including one of the CD4+ T cells and the CD8+ T cells and the second composition including the other of the CD4+ T cells or the CD8+ T cells.

The dose of bispecific polypeptide to be used for administration directly to a subject, or for exposure to immune cells in vitro, can be determined by a person skilled in the art and will typically depend on factors including, but not limited to, the type, size, stage and receptor status of the tumour in addition to the age, weight and general health of the subject. Another determinative factor may be the risk of developing recurrent disease. For instance, for a subject identified as being at high risk or higher risk or developing recurrent disease, a more aggressive therapeutic regimen may be prescribed as compared to a subject who is deemed at a low or lower risk of developing recurrent disease. Similarly, for a subject identified as having a more advanced stage of cancer, for example, stage III or IV disease, a more aggressive therapeutic regimen may be prescribed as compared to a subject that has a less advanced stage of cancer.

### Bispecific Polypeptides

As used herein, the term “bispecific polypeptide” refers a polypeptide that can specifically bind two different target antigens simultaneously. The bispecific polypeptides for use in the methods as described herein comprise two structurally distinct antigen binding domains or proteins (i.e., regions), each of which specifically binds to a single target antigen. Bispecific polypeptides can be used to bind to a target antigen on an APC and a different target antigen on an immune effector cell.

Preferably, the binding protein comprises an antigen binding protein for binding to a target antigen on a professional APC, more preferably, an endogenous professional APC of the subject requiring treatment. Examples of such antigens and such cells are further described herein.

Preferably the immune effector cell is an immune effector cell that expresses a heterologous receptor comprising an antigen binding domain and a signalling domain, such as a chimeric antigen receptor (CAR). Accordingly, in preferred embodiments, the bispecific polypeptide comprises an antigen binding domain for binding to an antigen on a CAR-expressing immune cell (such as a CAR T cell or a CAR NK cell).

It will also be appreciated that the bispecific polypeptides can be used to bind to a target antigen on an APC and a target antigen on a heterologous receptor (eg CAR) expressed by an immune cell.

The antigen binding proteins of the bispecific polypeptides for use according to the invention may comprise antibodies, or antigen binding fragments thereof. For example, the bispecific polypeptides can include polypeptide sequences (i.e., domains) of one of more antibodies or antibody fragments. Examples of antibody fragments are further described herein.

In any embodiment, the bispecific polypeptide for use in the methods as described herein, may comprise a tandem single-chain variable fragment antibody (taFv) having a first scFv and a second scFv.

The bispecific polypeptides for use in the methods as described herein may be variously referred to as “Bispecific Engagers of APCs and T cells” or “BEATs”.

The term “antigen presenting cell” or “APC” as used herein preferably refers to be a professional antigen presenting cell (e.g., a dendritic cell, macrophage, B-cell etc) as distinct from a non-professional antigen presenting cell (e.g., a fibroblast, thymic epithelial cell, thyroid epithelial cell, glial cell, pancreatic beta cell, vascular endothelial cell, etc.). In a preferred embodiment, the APC is a professional antigen binding cell preferably an endogenous professional APC of the subject requiring treatment. In certain embodiments, the endogenous professional APC is not a cell of the tumour or cancer that is being treated in the subject.

In any embodiment, the first antigen binding protein of the bispecific polypeptide, specifically binds to an APC antigen selected from the group consisting of MHCII, Clec9a, PD-L1, PD-L2, galectin, CD11c, CD19, CD40, CD206 (also known as mannose receptor) and CD83. MHCII is expressed on dendritic cells (DCs), mononuclear phagocytes, some endothelial cells, thymic epithelial cells, and B cells; Clec9a is expressed on BDCA+dendritic cells and a small subset of CD14+/CD16-monocytes; PD-L1 and PD-L2 are expressed on macrophages, myeloid DCs, B cells and vascular endothelial cells; galectin is expressed by T helper cells and B cells; CD11c is expressed at high levels by DCs; CD19 is expressed on all professional B cells to mature B cells and follicular DCs; CD40 is expressed by dendritic cells, B cells and macrophages; and CD83 is predominantly expressed by mature DCs. CD206 is primarily present on the surface of macrophages and immature dendritic cells. In any embodiment, the antigen specifically bound by the first binding protein is not a tumour-associated antigen.

In any embodiment, the second antigen binding protein of the bispecific polypeptide specifically binds to an antigen on an immune effector cell expressing a CAR or similar receptor. For example, the second binding protein of the bispecific polypeptides for use in the methods of the invention, may bind to an antigen on the immune cell, wherein the antigen is not part of the CAR, but is an antigen present on the cell surface of the immune cell. Alternatively, the second antigen binding protein may bind directly to the CAR expressed by the immune cell, such as to the antigen binding protein of the CAR, or an extracellular region of the CAR that is not involved in antigen binding, as further described below.

In certain embodiments, the CAR present on the immune cells may comprise additional amino acids or molecules for binding by the second antigen binding protein. As is known in the art, CAR constructs may be designed to include a “tag”, which is typically a short amino acid sequence that is specifically recognized by an antibody. In some embodiments, the immune cell is a T cell or a NK cell engineered to express a CAR which includes a tag. In the context of such embodiments, the second antigen binding protein of the bispecific polypeptide may bind to the tag or it may bind to a region of the CAR other than the tag.

Illustrative examples of tags which may be present on CARs include peptide tags (e.g., FLAG-tag, HA-tag, His-tag, Myc-tag, S-tag, SBP-tag, Strep-tag, exact-tag) and protein tags (e.g., GST-tag, MBP-tag, GFP-tag, a tag in the form of a protein for binding to an antibody or other protein, a nuclear protein (such as a leucine zipper) or any other protein modification to a CAR). In any embodiment, the antigen or affinity tag on the CAR is a c-Myc-tag or a FLAG-tag.

In some embodiments, the immune cell is a T cell or a NK cell engineered to express a CAR which does not include a tag. In some embodiments, the immune cell is a T cell or a NK cell engineered to express a CAR which does not include a tag or any heterologous tumour-associated antigens or fragments of tumour-associated antigens.

In certain embodiments, the immune cell is a T cell or a NK cell engineered to express a CAR and the bispecific polypeptide binds to an extracellular part of the CAR. The extracellular domain of the CAR comprises an antigen binding domain, typically for binding a tumour associated or tumour specific antigen. Typically, the antigen binding domain is an antibody fragment, such as a Fab or scFv. Most typically, the antigen binding domain is an scFv. The extracellular domain also typically comprises a spacer (or hinge) region linking the antigen-binding domain to the transmembrane domain. The spacer region may be derived from an immunoglobulin, such as IgG1 or IgG4, or it may be derived from alternative cell-surface proteins, including, but not limited to, CD4, CD8, or CD28.

Accordingly, in certain embodiments, the second antigen binding protein of the bispecific polypeptide for use in the methods of the invention may be for binding to the antigen binding domain of the CAR. As such, the second antigen binding protein of the bispecific polypeptide may be derived from an anti-idiotype antibody or antigen-binding fragment thereof, wherein the anti-idiotype antibody is an anti-idiotype antibody of the antibody portion of a CAR.

Anti-idiotype antibodies are known in the art and the skilled person will be well able to utilise to the antigen binding domains of these antibodies in the design of a bispecific polypeptide for use in the methods of the invention. Accordingly, in some embodiments, the second antigen binding domain of the bispecific polypeptide for use in the methods of the invention comprises an antibody or antibody fragment derived from an anti-idiotype antibody specific for an anti-CD 19, anti-Her2, anti-mesothelin or other antibody, or antigen-binding fragment of an anti-idiotype antibody for binding an antibody found in a CAR.

A number of anti-idiotype antibodies are known in the art. For example, International Patent Application Publication No. WO 2014/190273 and Jena et al. 2013, PLOS One, 8 (3): e57838, describe an anti-idiotype antibody (mAb clone no. 136.20.1) that recognizes the anti-CD19 scFv FMC63, which is used in a number of CAR constructs in current development.

In certain embodiments, the second antigen binding protein of the bispecific polypeptide for use in the methods of the invention comprises an antibody or antigen-binding protein derived from an anti-idiotype antibody specific for an anti-CD19 antibody, or antigen-binding fragment of the anti-idiotype antibody, that may have one or more of the same CDRs (i.e. one or more of, or all of, VH CDR1, VH CDR2, CH CDR3, VL CDR1, VL CDR2, and VL CDR3, using the Kabat definition, the Chothia definition, or a combination of the Kabat and Chothia definitions) as mAb clone no. 136.20.1. In some embodiments, the multi-specific antigen-binding construct comprises an antigen-binding polypeptide construct derived from an anti-idiotype antibody specific for an anti-CD19 antibody, or antigen-binding fragment of the anti-idiotype antibody, that may have one or more (for example, two) variable regions from mAb clone no. 136.20.1. In some embodiments, the second antigen binding protein comprises an antigen-binding polypeptide construct derived from an anti-idiotype antibody specific for an anti-CD19 antibody, or antigen-binding fragment of the anti-idiotype antibody, that binds to the same epitope as mAb clone no. 136.20.1.

Other examples of anti-idiotype antibodies include those that are commercially available from AbD Serotec®, an anti-idiotype antibody specific for an anti-CD22 antibody described in International Patent Publication No. WO 2013/188864, an anti-idiotype antibody specific for an anti-CEA antibody described in International Patent Publication No. WO 97/34636, an anti-idiotype antibody specific for an anti-GD2 antibody described in U.S. Pat. No. 5,935,821, and an anti-idiotype antibody specific for an anti-NY-ESO-1 antibody described in Jakka et al. 2013, Anticancer Research, 33 (10): 4189-420. Custom anti-idiotype antibodies may also be obtained from AbD Serotec®.

Alternatively, anti-idiotype antibodies to CARs targeting CD19 or other tumour-associated antigens may be made according to the method described in Jena et al., supra, and used for the construction of an anti-idiotype antigen-binding polypeptide construct.

In further embodiments, the bispecific polypeptide comprises a second antigen binding protein that binds to an extracellular region of a CAR that is not involved in antigen binding. For example, the second binding protein of the bispecific polypeptide may bind to the hinge region of the CAR (i.e., between the transmembrane domain and scFv portion of the CAR), or to the spacer region between the Variable Heavy and Variable Light chains of the scFv portion of the CAR, or any other region of the CAR.

In some embodiments, the hinge region may be an scFv-CD28 or scFv-CD8 junction, which comprises neo-epitopes that may be targeted by the second binding protein. In some embodiments, the hinge region may comprise mutated (Fe-binding null) IgG CH2/3 that may be targeted by the second binding protein. In some embodiments, the hinge region may comprise a spacer such as a Strep-tag II as described by Liu et al. (2016, Nature Biotechnology, 34:430-434) that may be targeted by the second binding protein.

An example of an anti-CAR antibody that binds to a hinge region of the CAR molecule is the 2D3 antibody described in International Patent Application Publication No. WO 2014/190273, which binds to an IgG4 CH2-CH3 hinge region. In some embodiments, the multi-specific antigen-binding construct comprises an antigen-binding polypeptide construct that binds to an IgG4 CH2-CH3 hinge region. In some embodiments, the multi-specific antigen-binding construct comprises an antigen-binding polypeptide construct that binds to an IgG4 CH2-CH3 hinge region and has one or more of the same CDRs (i.e., one or more of, or all of, VH CDR1, VH CDR2, CH CDR3, VL CDR1, VL CDR2 and VL CDR3) as 2D3, or has one or more (for example, two) variable regions of 2D3 as described in WO 2014/190273. In some embodiments, the second binding protein of the bispecific polypeptide binds to an IgG4 CH2-CH3 hinge region and binds to the same epitope as 2D3 as described in WO 2014/190273.

In further embodiments, the second antigen binding protein of the bispecific polypeptide binds to an antigen on an immune cell that expresses a CAR, wherein the antigen is not the CAR or is not on the CAR. For example, the antigen may comprise a naturally-occurring protein present on the immune cell surface (such as CD3, CD4, CD8, CD25, CD127, CD196 (CCR6), CD27, CD28, CD45RA, CD45RO, CD62L, CD197, and HLA-DR). Alternatively, the antigen on the immune cell may be an artificially introduced antigen, such as an affinity tag. Examples of peptide tags which may be present on the surface of the immune cell include peptide tags (e.g., FLAG-tag, HA-tag, His-tag, Myc-tag, S-tag, SBP-tag, Strep-tag, exact-tag) and protein tags (e.g., GST-tag, MBP-tag, GFP-tag).

The first and second antigen binding proteins of the bispecific polypeptide for use in the methods of the invention may be in the form of an antibody or an antibody fragment. In one embodiment, the first antigen binding protein comprises a scFv. In another embodiment, the second antigen binding protein comprises a scFv. In certain embodiments, the first and second antigen binding proteins are different antibody or antibody fragment formats. Alternatively, the first and second antigen binding proteins may be the same antibody or antibody fragment formats. In certain examples, the first antigen binding protein (ie for binding to an antigen on an APC, preferably professional APC), is in the form of an immunoglobulin (such as IgG1, IgG2, IgG3, or IgG4) and the second antigen binding protein is in the form of an antibody fragment (such as an scFv). In further examples, the second antigen binding protein (ie for binding to an antigen on an immune cell, preferably CAR T cell), is in the form of an immunoglobulin (such as IgG1, IgG2, IgG3, or IgG4) and the first antigen binding protein (for binding an antigen on the APC) is in the form of an antibody fragment (such as an scFv).

In preferred embodiments, the bispecific polypeptides for use in accordance with the present invention are in the form of fusion proteins. The fusion proteins preferably comprise the first antigen binding protein, joined to the second antigen binding protein, either directly, or via a linker. The linker may be any linker known to the skilled person for use in the context of a fusion protein.

The linkers may, for example, function to join two proteins of an antigen-binding polypeptide construct (such as the VH and VL of an scFv or diabody), or they may function to join two antigen-binding polypeptide constructs together (such as two or more Fabs or sdAbs), or they may function to join an antigen-binding polypeptide construct to a scaffold. In some embodiments, the bi-specific polypeptide may comprise multiple linkers (i.e., two or more), for example, one or more scFvs linked to a scaffold may comprise a linker joining the VH and VL of the scFv and a linker joining the scFv to the scaffold. Appropriate linkers are known in the art and can be readily selected by the skilled artisan based on the intended use of the linker (see, for example, Miller & Kontermann, “Bispecific Antibodies” in Handbook of Therapeutic Antibodies, Wiley-VCR Verlag GmbH & Co. 2014).

Useful linkers include glycine-serine (GlySer) linkers, which are well-known in the art and comprise glycine and serine units combined in various orders. Examples include, but are not limited to, (GS), (GSGGS)n, (GGGS)n and (GGGGS)n, where n is an integer of at least one, typically an integer between 1 and about 10, for example, between 1 and about 8, between 1 and about 6, or between 1 and about 5.

Other useful linkers include sequences derived from immunoglobulin hinge sequences. The linker may comprise all or part of a hinge sequence from any one of the four IgG classes and may optionally include additional sequences. For example, the linker may include a portion of an immunoglobulin hinge sequence and a glycine-serine sequence. A nonlimiting example is a linker that includes approximately the first 15 residues of the IgG 1 hinge followed by a GlySer linker sequence, such as those described above, that is about 10 amino acids in length.

The length of the linker will vary depending on its application. Appropriate linker lengths can be readily selected by the skilled person. For example, when the linker is to connect the VH and VL domains of an scFv, the linker is typically between about 5 and about 20 amino acids in length, for example, between about 10 and about 20 amino acid in length, or between about 15 and about 20 amino acids in length. When the linker is to connect the VH and VL domains of a diabody, the linker should be short enough to prevent association of these two domains within the same chain. For example, the linker may be between about 2 and about 12 amino acids in length, such as, between about 3 and about 10 amino acids in length, or about 5 amino acids in length.

In some embodiments, when the linker is to connect two Fab fragments, the linker may be selected such that it maintains the relative spatial conformation of the paratopes of a F(ab′) fragment, and is capable of forming a covalent bond equivalent to the disulphide bond in the core hinge of IgG. In this context, suitable linkers include IgG hinge regions such as, for example those from IgG1, IgG2 or IgG4. Modified versions of these exemplary linkers can also be used. For example, modifications to improve the stability of the IgG4 hinge are known in the art (see for example, Labrijn et al. 2009, Nature Biotechnology, 27:767-771).

The linker may comprise a sequence of amino acid residues joining the first and second antigen binding proteins. Alternatively, the first and second binding proteins may be linked via chemical conjugation (for example, to form a bis-aryl conjugate between the proteins). Examples of suitable methods for chemical conjugation of binding proteins are known in the art. Such methods include the use of succinimidyl compound modification of primary amines present on lysine residues, as used in TriLink Technologies bioconjugation reagents. In an embodiment, if the first and/or second binding proteins are scFvs, the bispecific polypeptide comprises a linker sequence between the VH and VL proteins of the scFvs. Suitable linker sequences would be known to persons skilled in the art, illustrative examples of which include relatively flexible and hydrophilic amino acid residues.

It will be within the purview of the skilled person to determine the appropriate architecture of the bispecific polypeptides for use according to the invention.

In any embodiment, the first and/or second binding domain may be in the form of an antibody or antigen binding fragment thereof. The antigen binding protein may be an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein. As used herein the antigen binding protein may be a variable domain.

The first and second antigen binding proteins of the bispecific polypeptide are preferably in the form of an antibody, or antigen binding fragment thereof. In any embodiment, the first and/or second antigen binding proteins are in the form of an antibody, such as an immunoglobulin G (IgG1, IgG2, IgG3, or IgG4). Optionally, the first antigen binding protein is in the form of an antibody, such as an immunoglobulin G (IgG1, IgG2, IgG3, or IgG4) and the second antigen binding protein is in the form of an antibody fragment (such as an scFv, a dimeric scFv (di-scFv), a Fab, a Fv, a F(ab′)2), wherein the second antigen binding protein is linked to the first antigen binding protein via the C or N terminus, or via an internal region of the first antigen binding protein, preferably wherein the second antigen binding protein is linked to the first antigen binding protein via the C terminus of the heavy chain of the first antigen binding protein. Optionally, the second antigen binding proteins is in the form of an antibody, such as an immunoglobulin G (IgG1, IgG2, IgG3, or IgG4) and the first antigen binding protein is in the form of an antibody fragment (such as an scFv, a dimeric scFv (di-scFv), a Fab, a Fv, a F(ab′)2), wherein the first antigen binding protein is linked to the second antigen binding protein via the C or N terminus, or an internal region of the second antigen binding protein, preferably wherein the second antigen binding protein is linked to the second antigen binding protein via the C terminus of the heavy chain of the second antigen binding protein.

In any embodiment, the bispecific polypeptides for use according to the invention can contain additional amino acids or molecules for purification or identification. For example, the polypeptide can contain an epitope or affinity tag. Illustrative examples of such epitopes or affinity tags include, peptide tags (e.g., FLAG-tag, HA-tag, His-tag, Myc-tag, S-tag, SBP-tag, Strep-tag, eXact-tag) and protein tags (e.g., GST-tag, MBP-tag, GFP-tag). In an embodiment, the epitope or affinity tag is a His-tag.

In any embodiment, the bispecific polypeptide comprises a first antigen binding protein for binding to CD40. In any embodiment, the bispecific polypeptide comprises a second antigen binding protein for binding to a FLAG tag.

In any embodiment, the bispecific polypeptide comprises a first antigen binding protein and a second antigen binding protein, wherein the first antigen binding protein specifically binds to CD40 and wherein the second antigen binding protein specifically binds to a FLAG tag.

Preferably, the first antigen binding protein binds the outer A-module of the membrane proximal domain (D1a) of CD40, optionally wherein said binding does not prevent, reduce or inhibit CD40-CD40L binding.

In an embodiment the first antigen binding protein comprises an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 in relation to CD40 binding proteins.

In any embodiment, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

Reference herein to a protein or antibody that “binds to” CD40 provides literal support for a protein or antibody that “binds specifically to” or “specifically binds to” CD40.

In any embodiment, the first antigen binding protein competitively inhibits the binding to CD40 of an antibody or antigen binding fragment thereof, comprising a VH comprising a sequence as set forth in SEQ ID NO: 1 and a VL comprising a sequence as set forth in SEQ ID NO: 2.

In any embodiment the first antigen binding protein comprises a CDRH1, a CDRH2 and/or a CDRH3 of an antigen binding domain having a variable heavy domain as defined in SEQ ID NO: 1.

In any embodiment the first antigen binding protein comprises a CDRL1, a CDRL2 and/or a CDRL3 of an antigen binding domain having a variable light chain as defined in of SEQ ID NO: 2.

In any embodiment of the first antigen binding protein comprises a CDR1, a CDR2 and/or a CDR3 of an antigen binding domain having a variable heavy chain as defined in SEQ ID NO: 1 and a variable light chain as defined in SEQ ID NO: 2.

In any embodiment the first antigen binding protein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1 below.

As defined herein, the linker may be a chemical, one or more amino acids, or a disulphide bond formed between two cysteine residues.

In any embodiment the first antigen binding protein comprises:
(i) a VH comprising a complementarity determining region (CDR) 1 comprising or consisting of a sequence as set forth in SEQ ID NO: 3, a CDR2 comprising or consisting of a sequence as set forth in SEQ ID NO: 4 and a CDR3 comprising or consisting of a sequence as set forth in SEQ ID NO: 5, and
(ii) a VL comprising a complementarity determining region (CDR) 1 comprising or consisting of a sequence as set forth in SEQ ID NO: 6, a CDR2 comprising or consisting of a sequence as set forth in SEQ ID NO: 7 and a CDR3 comprising or consisting of a sequence as set forth in SEQ ID NO: 8;
or
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 3, a CDR2 comprising or consisting of a sequence at least about 80%, at least 85%, at least 90%, at least 92%, at least 95%, at least 97%, at least 99% identical to a sequence set forth in SEQ ID NO: 4; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 5, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 6, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 7, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8.

In any embodiment, the first antigen binding protein comprises:
(i) a VH comprising a complementarity determining region (CDR) 1 comprising or consisting of a sequence as set forth in SEQ ID NO: 9, a CDR2 comprising or consisting of a sequence as set forth in SEQ ID NO: 10 and a CDR3 comprising or consisting of a sequence as set forth in SEQ ID NO: 11, and
(ii) a VL comprising a complementarity determining region (CDR) 1 comprising or consisting of a sequence as set forth in SEQ ID NO: 12, a CDR2 comprising or consisting of a sequence as set forth in SEQ ID NO: 13 and a CDR3 comprising or consisting of a sequence as set forth in SEQ ID NO: 8;
or
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 9, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10; a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 11, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 13, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8.

In any embodiment, FR1, FR2, FR3 and FR4 may refer to framework regions from the variable heavy chain of an antibody (a VH), FR1a, FR2a, FR3a and FR4a may refer to framework regions from the variable light chain of an antibody (a VL), or where FR1, FR2, FR3 and FR4 are framework regions from the VL, FR1a, FR2a, FR3a and FR4a are framework regions from VH. In such examples, the FRs may be referred to as FR H1, FR H2, FR H3, FR H4, FR L1, FR L2, FR L3 and FR L4 as the case may be.

In an embodiment, the second antigen binding protein comprises an antigen binding domain with a FR H1, a FR H2, a FR H3 and/or a FR H4 of an antigen binding domain having a variable heavy chain as defined in SEQ ID NO: 1 (according to Kabat or IMGT numbering).

In any embodiment, the second antigen binding protein comprises an antigen binding domain with a FR L1, a FR L2, a FR L3 and/or a FR L4 of an antigen binding domain having a variable light chain as defined in SEQ ID NO: 2 (according to Kabat or IMGT numbering).

In any embodiment, the second antigen binding protein comprises an antigen binding domain comprising a FR1, a FR2, a FR3 and/or a FR4 of an antigen binding domain having a variable heavy chain as defined in SEQ ID NO: 1 (according to Kabat or IMGT numbering), and a FR1, a FR2, a FR3 and/or a FR4 of an antigen binding domain having a variable light chain as defined in SEQ ID NO: 2 (according to Kabat or IMGT numbering).

In any embodiment the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 1; optionally wherein the amino acid substitutions are not in the CDRs and/or the antigen binding protein retains the ability to bind to CD40.

In any embodiment the first antigen binding protein comprises a light chain variable domain comprising or consisting of the amino acid sequence as set forth in SEQ ID NO: 2; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 2; optionally wherein the amino acid substitutions are not in the CDRs and/or the antigen binding protein retains the ability to bind to CD40.

In any embodiment, the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto, and a light chain variable domain comprising or consisting of the amino acid sequence as set forth in SEQ ID NO: 2, or sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy and/or light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 1 or 2, respectively; optionally wherein the amino acid substitutions are not in the CDRs and/or the antigen binding protein retains the ability to bind to CD40.

In any embodiment the first antigen binding protein comprises, consists essentially of or consists of the amino acid sequence of (in order of N to C terminus or C to N terminus) SEQ ID NO: 1 and 2.

In any embodiment, the first antigen binding protein may have from N to C terminus a VH then VL, or a VL then VH, or any CDR 1, 2 and 3 defined herein as VH then any CDR 1, 2 and 3 defined herein as VL, or any CDR 1, 2 and 3 defined herein as VL then any CDR 1, 2 and 3 defined herein as VH.

In any embodiment, the first antigen binding protein may be in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

Further, as described herein, in any embodiment, the first antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

In any embodiment, the first antigen binding protein may be in the form of an immunoglobulin G molecule (IgG). Optionally, in accordance with any embodiment of the first aspect of the invention, the first antigen binding protein may be in the form of an IgG or heterodimeric Fab-Fc, and the second antigen binding protein may be in the form of an antigen binding fragment of an IgG, such as an scFv.

In any embodiment, the second antigen binding protein is for binding to a FLAG tag, the second antigen binding protein comprising an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 herein.

In any embodiment, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

In any embodiment, the second antigen binding protein is capable of specifically binding to a FLAG tag or variant thereof (such as defined in SEQ ID NOs: 28 or 30 or as otherwise defined herein). The antigen binding protein is preferably capable of specifically binding to protein domains comprising multiples of the FLAG tag sequences (eg 2×FLAG, 3×FLAG etc).

In any embodiment, the second antigen binding protein is for specifically binding to a peptide tag that contains or consists of the sequence DYK, preferably the sequence DYKD (SEQ ID NO: 28). In addition to these sequences, other amino acids can be present in the FLAG tag bound by the second antigen binding protein, preferably wherein the additional amino acids in the FLAG tag are hydrophilic amino acids for example R (Arg), D (Asp), E (Glu) and K (Lys) and/or amino acids with aromatic side chains for example Y (Tyr), F (Phe), H (His) and W (Trp).

In preferred embodiments the second antigen binding protein is capable of specifically binding to a FLAG-tag that contains, comprises or consists of the sequence GDYKDDDDKG (SEQ ID NO: 29), DYKDDDDK (SEQ ID NO: 30), MDYKDDDDK (SEQ ID NO: 31), DFKDDDK (SEQ ID NO: 32), DYKAFDNL (SEQ ID NO: 33), DYKDHDG (SEQ ID NO: 34), MDFKDDDDK (SEQ ID NO: 35), MDYKAFDNL (SEQ ID NO: 36), DYKDHDI (SEQ ID NO: 37), DYKDH (SEQ ID NO: 38), DYKDD (SEQ ID NO: 39), DYKDHD (SEQ ID NO: 40) and/or DYKDDD (SEQ ID NO: 41). The most preferred sequence is DYKDDDDK (SEQ ID NO: 30).

As used herein, the term FLAG-tag also encompasses modified FLAG-tags, such as those derived from the FLAG-tags described above, especially the tag with the sequence DYKDDDDK, by amino acid insertion, deletion or substitution.

Reference herein to a protein or antibody that “binds to” FLAG tag provides literal support for a protein or antibody that “binds specifically to” or “specifically binds to” a FLAG tag.

In any embodiment, the FLAG tag is present at the N-terminus, the C-terminus or within the protein to which the bispecific polypeptide of the invention is capable of binding to, binds to or specifically binds to.

In any embodiment, the second antigen binding protein competitively inhibits the binding to a FLAG tag of an antibody comprising a VH comprising a sequence as set forth in SEQ ID NO: 15 and a VL comprising a sequence as set forth in SEQ ID NO: 16.

In any embodiment, the second antigen binding protein comprises a CDRH1, a CDRH2 and/or a CDRH3 of an antigen binding domain having a variable heavy chain as defined in SEQ ID NO: 15.

In any embodiment, the second antigen binding protein comprises a CDRL1, a CDRL2 and/or a CDRL3 of an antigen binding domain having a variable light chain as defined in SEQ ID NO: 16.

In any embodiment, the second antigen binding protein comprises a CDR1, a CDR2 and/or a CDR3 of an antigen binding domain having a variable heavy chain as defined in SEQ ID NO: 15 and a variable light chain as defined SEQ ID NO: 16.

In any embodiment the second antigen binding protein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1.

As defined herein, the linker may be a chemical, one or more amino acids, or a disulphide bond formed between two cysteine residues.

In any embodiment, the second antigen binding domain comprises an antigen binding domain comprising:
(a) a VH comprising a CDR1 comprising or consisting of an amino acid sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence as set forth in SEQ ID NO: 17; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 18; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 19, and
(b) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 20; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 21; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 22;
or
(c) a VH comprising a complementarity determining region CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 23; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 24; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 25, and
(d) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 26; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 27; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 22;

In any embodiment, the second antigen binding protein comprises an antigen binding domain comprising:
(a) a VH comprising a CDR1 comprising or consisting of an amino acid sequence of SEQ ID NO: 17; a CDR2 comprising or consisting of an amino acid sequence of SEQ ID NO: 18; a CDR3 comprising or consisting of an amino acid sequence of SEQ ID NO: 19, and
(b) a VL comprising a CDR1 comprising or consisting of an amino acid sequence SEQ ID NO: 20; a CDR2 comprising or consisting of an amino acid sequence of SEQ ID NO: 21; a CDR3 comprising or consisting of an amino acid sequence SEQ ID NO: 22;
or
(c) a VH comprising a CDR1 comprising or consisting of an amino acid sequence of SEQ ID NO: 23; a CDR2 comprising or consisting of an amino acid sequence of SEQ ID NO: 24; a CDR3 comprising or consisting of an amino acid sequence of SEQ ID NO: 25, and
(d) a VL comprising a CDR1 comprising or consisting of an amino acid sequence of SEQ ID NO: 26; a CDR2 comprising or consisting of an amino acid sequence of SEQ ID NO: 27; a CDR3 comprising or consisting of an amino acid sequence of SEQ ID NO: 22.

In any embodiment, FR1, FR2, FR3 and FR4 may refer to framework regions from the variable heavy chain of an antibody (a VH), FR1a, FR2a, FR3a and FR4a may refer to framework regions from the variable light chain of an antibody (a VL), or where FR1, FR2, FR3 and FR4 are framework regions from the VL, FR1a, FR2a, FR3a and FR4a are framework regions from VH. In such examples, the FRs may be referred to as FR H1, FR H2, FR H3, FR H4, FR L1, FR L2, FR L3 and FR L4 as the case may be.

In any embodiment, the second antigen binding protein comprises an antigen binding domain with a FR H1, a FR H2, a FR H3 and/or a FR H4 from a human germline, wherein the human germline is IGHV1-46*01 or IGHV7-4-1*02.

In any embodiment, the second antigen binding protein comprises an antigen binding domain with a FR L1, a FR L2, a FR L3 and/or a FR L4 from a human germline, wherein the human germline is IGKV2-30*01 or IGKV4-1*01.

In any embodiment of, the second antigen binding protein comprises an antigen binding domain with a FR H1, a FR H2, a FR H3 and/or a FR H4 from a human germline, wherein the human germline is IGHV1-46*01 or IGHV7-4-1*02, and a FR L1, a FR L2, a FR L3 and/or a FR L4 from a human germline, wherein the human germline is IGKV2-30*01 or IGKV4-1*01.

In an embodiment, the second antigen binding protein comprises an antigen binding domain with a FR H1, a FR H2, a FR H3 and/or a FR H4 of an antigen binding domain having a variable heavy chain as defined in SEQ ID NO: 15 (according to Kabat or IMGT numbering).

In any embodiment, the second antigen binding protein comprises an antigen binding domain with a FR L1, a FR L2, a FR L3 and/or a FR L4 of an antigen binding domain having a variable light chain as defined in SEQ ID NO: 16 (according to Kabat or IMGT numbering).

In any embodiment, the second antigen binding protein comprises an antigen binding domain comprising a FR1, a FR2, a FR3 and/or a FR4 of an antigen binding domain having a variable heavy chain as defined in SEQ ID NO: 15 (according to Kabat or IMGT numbering), and a FR1, a FR2, a FR3 and/or a FR4 of an antigen binding domain having a variable light chain as defined in SEQ ID NO: 16 (according to Kabat or IMGT numbering).

In any embodiment, the second antigen binding protein comprises an antigen binding domain comprising a variable heavy chain comprising the amino acid sequence as set forth in SEQ ID NO: 15, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 15; optionally wherein the amino acid substitutions are not in the CDRs and/or the antigen binding protein retains the ability to bind to FLAG tag.

In any embodiment, the second antigen binding protein comprises an antigen binding domain comprising a variable light chain comprising the amino acid sequence as set forth in SEQ ID NO: 16; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the light chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 16; optionally wherein the amino acid substitutions are not in the CDRs and/or the antigen binding protein retains the ability to bind to FLAG tag.

In any embodiment, the second antigen binding protein comprises an antigen binding domain comprising a variable heavy chain comprising the amino acid sequence as set forth in SEQ ID NO: 15 and a variable light chain comprising the amino acid sequence as set forth in SEQ ID NO: 16; or sequences at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy and light chain variable domains of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 15 and 16, respectively; optionally wherein the amino acid substitutions are not in the CDRs and/or the antigen binding protein retains the ability to bind to FLAG tag.

As described herein, the second antigen binding protein may be in the form of:
(i) a single domain antibody (sdAb);
(ii) a single chain Fv fragment (scFv);
(iii) a dimeric scFv (di-scFv);
(iv) one of (ii) or (iii) linked to a constant region of an antibody, Fc or a heavy chain constant domain (CH) 2 and/or CH3;
(v) one of (i) to (iv) linked to a protein that binds to an immune effector cell;
(vi) one of (i) to (iv) linked to a modified immune cell receptor, such as a modified T cell receptor.

Further, as described herein, the second antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

In preferred embodiments, the second antigen binding protein is in the form of an scFv and the first antigen binding protein is in the form of an immunoglobulin G (IgG) antibody.

In any embodiment of any aspect of the invention, the bispecific polypeptide comprises, consists essentially of or consists of an amino acid sequence of SEQ ID NOs: 42 and 43.

As used herein, the complementarity determining region sequences (CDRs) of any antigen binding protein referred to herein, are defined according to the IMGT, Chothia or Kabat numbering system or any other CDR numbering system.

### Nucleic Acids and Vectors

In another aspect disclosed herein, there is provided a nucleic acid encoding the bispecific polypeptide for use in the methods as described herein. In yet another aspect there is provided a cell comprising the vector for use in the methods as described herein. For example, it will be appreciated that in certain embodiments, the bispecific polypeptide is not administered directly to the subject requiring treatment, and may instead be expressed in vivo in the subject, from a nucleic acid sequence or construct provided in the subject.

The terms “polynucleotide”, “polynucleotide sequence”, “nucleotide sequence”, “nucleic acid” or “nucleic acid sequence” as used interchangeably herein to designate mRNA, RNA, CRNA, cDNA or DNA. The term typically refers to polymeric form of nucleotides of at least 10 bases in length, either ribonucleotides or deoxynucleotides or a modified form or either type of nucleotide. The term includes single and double stranded forms of RNA and DNA.

In another aspect, there is provided a vector comprising the nucleic acid described herein operably linked to a regulatory sequence.

The terms “regulatory element” or “regulatory sequence” refer to nucleic acid sequences (e.g., DNA) necessary for expression of an operably linked coding sequence in a particular cell. The regulatory sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

Typically, the regulatory sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated. The promoters may be either naturally occurring promoters, or hybrid promoters that combine elements of more than one promoter.

Promoter sequences contemplated may be native to mammalian cells or may be derived from an alternative source, where the region is functional in the chosen organism. The choice of promoter will differ depending on the intended host cell. For example, promoters which could be used for expression in mammalian cells include the metallothionein promoter, which can be induced in response to heavy metals such as cadmium, the □-actin promoter as well as viral promoters such as the SV 40 large T antigen promoter, human cytomegalovirus (CMV) immediate early (IE) promoter, Rous sarcoma virus LTR promoter, the mouse mammary tumour virus LTR promoter, the adenovirus major late promoter (Ad MLP), the herpes simplex virus promoter, and a HPV promoter, particularly the HPV upstream regulatory region (URR), among others. All these promoters are well described in the art and readily available.

Enhancer elements may also be used herein to increase expression levels of the nucleic acid sequence within the vector construct. Examples include the SV40 early gene enhancer, as described for example in Dijkema et al. (1985, EMBO Journal, 4:761), the enhancer/promoter derived from the long terminal repeat (LTR) of the Rous Sarcoma Virus, as described for example in Gorman et al., (1982, Proceedings of the National Academy of Science. USA, 79:6777) and elements derived from human CMV, as described for example in Boshart et al. (1985, Cell, 41:521), such as elements included in the CMV intron A sequence.

The vector construct may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nts and may contain transcriptional and translational termination sequences in addition to a polyadenylation signal and any other regulatory signals capable of effecting mRNA processing or gene expression.

The term “operably connected” or “operably linked” as used herein refers to a juxtaposition wherein the components so described are in a relationship permitting them to function in their intended manner. For example, a regulatory sequence “operably linked” to a coding sequence refers to the positioning and/or orientation of the regulatory sequence relative to the coding sequence to permit expression of the coding sequence under conditions compatible with the regulatory sequence.

Recombinant nucleic acid molecules or polynucleotides may be inserted into a vector. Non-viral vectors such as plasmid expression vectors or viral vectors may be used. The kinds of vectors and the technique of insertion of the nucleic acid construct for use according to the methods of the invention are known in the art, illustrative examples of which include cosmids, plasmids (e.g., naked or contained in liposomes) and viruses (e.g., lentiviruses, retroviruses, adenoviruses, and adeno-associated viruses).

### Methods for Producing Bispecific Polypeptides

The bispecific polypeptides for use in the methods as described herein can be produced using any number of expression systems would be known to persons skilled in the art, illustrative examples of which include production in or by bacteria (e.g., E. coli, P. mirabilis), fungi (e.g., S. cerevisiae, P. pastoria, T. reesei), plants or plant cells, insects or insect cells (e.g., SF-9, SF21, Hi-5), or mammalian cells. In an embodiment, the expression system is a mammalian expression system. Suitable mammalian expression systems would be known to persons skilled in the art, illustrative examples of which include a CHO or 293 expression system. These expression systems are widely available from commercial suppliers. In an embodiment, the bispecific polypeptides are produced using a mammalian expression system.

A chemically conjugated bispecific polypeptide may be formed following succinimidyl compound modification of primary amines present on lysine residues, as described elsewhere herein.

It will be within the purview of the skilled person to utilise commercially available antibodies, or antigen-binding domains thereof, in order to generate the bispecific polypeptides for use in the methods as described herein.

Further, it is within the purview of the skilled person to reproduce antibodies or antigen binding proteins thereof based on published sequence information pertaining to antibodies or antigen binding proteins having the desired specificity, and to thereby include such antibodies or antigen binding proteins thereof in a bispecific polypeptide for use according to the methods of the present invention. In particular, it will be understood that the methods of the present invention encompasses the generation of any bispecific polypeptide which is designed to have the specific binding affinities of the first and second antigen binding proteins as described herein (i.e., a first binding protein for binding to an antigen on an antigen presenting cell, preferably a professional antigen presenting cell such as a dendritic cell; and a second binding protein for binding to an antigen on an immune cell expressing a CAR, including wherein the antigen is an antigen on the CAR). The skilled person will appreciate that any combination of antigen binding proteins with the desired binding specificity can be utilized to obtain a bispecific polypeptide of the invention.

Further, it is within the skill set of the skilled person to assess the binding capacity of any bispecific polypeptide made for use in accordance with the methods of the prior art. For example, methods for assessing binding to a protein are known in the art, e.g., as described in Scopes (In: Protein purification: principles and practice, Third Edition, Springer Verlag, 1994). Such a method generally involves immobilising the antigen binding protein and contacting it with labelled antigen. Following washing to remove non-specific bound protein, the amount of label and, as a consequence, bound antigen is detected. Of course, the antigen binding protein can be labelled and the antigen immobilised. Panning-type assays can also be used. Alternatively, or additionally, surface plasmon resonance assays can be used.

Optionally, the dissociation constant (Kd), association constant (Ka) and/or affinity constant (KD) of an immobilised antigen binding protein is determined. The “Kd” or “Ka” or “KD” for a bispecific polypeptide for use according to the invention is in one example measured by a radiolabelled or fluorescently-labelled ligand binding assay. In the case of a “Kd”, this assay equilibrates the antigen binding protein with a minimal concentration of labelled antigen in the presence of a titration series of unlabelled antigen. Following washing to remove unbound antigen, the amount of label is determined, which is indicative of the Kd of the protein.

According to another example the K_{d}, Kₐ or K_{D} is measured by using surface plasmon resonance assays, e.g., using BIAcore surface plasmon resonance (BIAcore, Inc., Piscataway, NJ) with immobilised antigen or immobilised antigen binding protein.

### Pharmaceutical Compositions

In another aspect, there is provided a pharmaceutical composition comprising the bispecific polypeptide described herein and a pharmaceutically acceptable carrier, for use in accordance with any method of the invention.

The compositions described for use herein may be prepared in a manner known in the art and are those suitable for parenteral administration to mammals, particularly humans, comprising a therapeutically effective amount of the composition with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions for use in methods of the invention may also include other supplementary physiologically active agents.

The carrier is typically pharmaceutically “acceptable” in the sense of being compatible with the other ingredients in the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the isolated T cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and non-aqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The present disclosure also contemplates the combination of the composition described herein with other active agents and/or in addition to other treatment regimens or modalities such as radiation therapy or surgery. When the composition described herein is used in combination with known active agents, the combination may be administered either in sequence (either continuously or broken up by periods of no treatment) or concurrently or as an admixture. Suitable anti-cancer agents will be known to persons skilled in the art. Treatment in combination is also contemplated to encompass the treatment with either the composition for use in the methods of the invention followed by a known treatment, or treatment with a known agent followed by treatment with the composition for use in the methods of the invention, for example, as maintenance therapy. For example, in the treatment of cancer it is contemplated that the composition for use in the methods of the present invention may be administered in combination with an alkylating agent (such as mechlorethamine, cyclophosphamide, chlorambucil, ifosfamidecysplatin, or platinum-containing alkylating agents such as cisplatin, carboplatin and oxaliplain), and anti-metabolite (such as a purine or pyrimidine analogue or an anti-folate agent, such as azathioprine and mercaptopurine), an anthracycline (such as daunorubicin, doxorubicin, epirubicin idarubicin, valrubicin, mitoxantrone or anthracycline analog), a plant alkaloid (such as a vinca alkaloid or a taxane, such as vincristine, vinblastine, vinorelbine, vindesine, paclitaxel or doestaxel), a topoisomerase inhibitor (such as a type I or type II topoisomerase inhibitor), a podophyllotoxin (such as etoposide or teniposide), a tyrosine kinase inhibitor (such as imatinib mesylate, nilotinib or dasatinib), an adenosine receptor inhibitor (such as A2aR inhibitors, SCH58261, CPI-444, SYN115, ZM241385, FSPTP or A2BR inhibitors such as PSB-1115), adenosine receptor agonists (such as CCPA, IB-MECA and CI-IB-MECA), a checkpoint inhibitor, including those of the PDL-1: PD-1 axis, nivolumab, pembrolizumab, atezolizumab, BMS-936559, MEDI4736, MPDL33280A or MSB0010718C), an inhibitor of the CTLA-4 pathway (such as ipilimumab and tremelimumab), an inhibitor of the TIM-3 pathway or an agonist monoclonal antibody that is known to promote T cell function (including anti-OX40, such as MEDI6469; and anti-41BB, such as PF-05082566).

In a further aspect, there is provided a kit or article of manufacture for use in the methods of the invention, wherein the kit or article includes one or more bispecific polypeptides of the invention, a nucleic acid encoding said bispecific polypeptide and/or pharmaceutical compositions as described above.

In further aspects there is provided a kit for use in the methods of the invention, the kit comprising:
(a) a container holding a bispecific polypeptide, nucleic acid, vector or pharmaceutical composition of the invention; and
(b) a label or package insert with instructions for use.

The kit may also comprise one or more active principles or ingredients for treatment of cancer. For example, the kit may also comprise an immune cell expressing a CAR.

The “kit” or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (e.g., the container may be an intravenous solution bag or a vial having a stopper pierceable by a hypodermic injection needle). The label or package insert indicates that the therapeutic composition is used for treating the condition of choice. In an embodiment, the label or package insert includes instructions for use and indicates that the therapeutic or prophylactic composition can be used to treat a cancer or other condition described herein.

The kit may comprise (a) a therapeutic or prophylactic composition; and (b) a second container with a second active principle or ingredient contained therein. The kit according to this embodiment for use in the methods of the invention may further comprise a package insert indicating the composition and other active principle can be used to treat cancer or condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further comprise other materials desirable from a commercial and user standpoint, which would be known to persons skilled in the art, suitable examples of which include other buffers, diluents, filters, needles, and syringes.

### EXAMPLES

### Example 1—CAR T Cell Multiple Stimulations with Either Bispecific BEAT and MoDCs or tumour

A bispecific polypeptide comprising a first antigen binding protein for binding to CD40 and a second antigen binding protein for binding to FLAG tag (ie an anti-CD40/anti-FLAG BEAT) was obtained according to the methods and disclosures herein. The amino acid sequence of the BEAT is provided in SEQ ID NO: 42 and 43.

CAR T cells were obtained by transducing CD3+ T cells with a nucleic acid sequence encoding a CAR comprising a FLAG-tag.

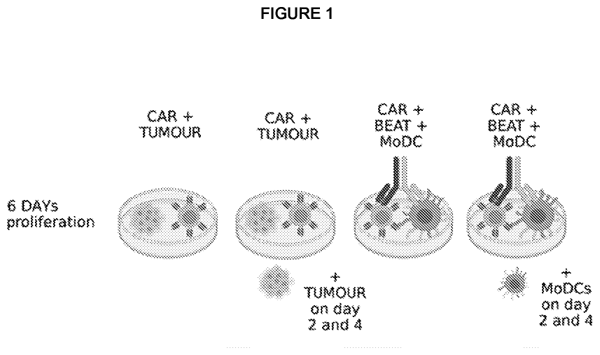

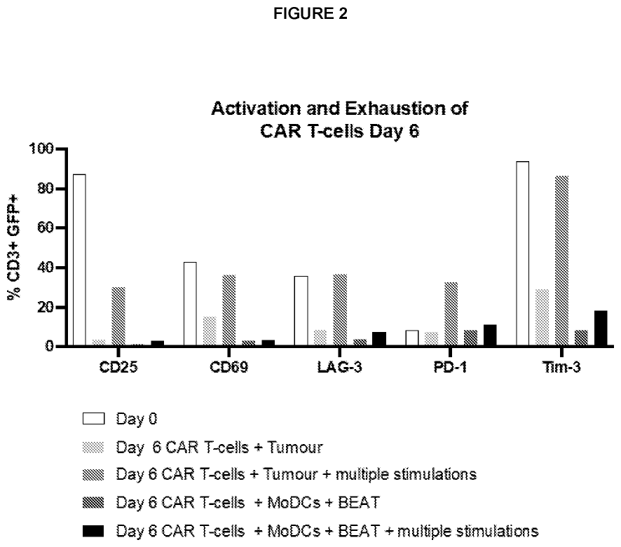

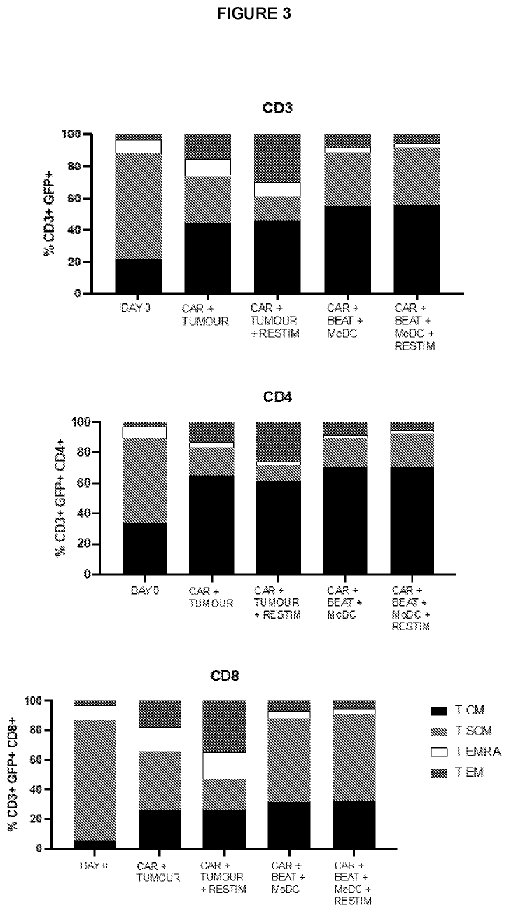

The CAR T cells were stimulated with either highly expressing mesothelin positive tumour cells or BEAT and monocyte derived DCs (MoDCs) (FIG. 1). Additional stimulant (tumour or MoDCs) was added on days 2 and 4 to each condition. Activation and exhaustion markers were measured at day 6 (FIG. 2). Phenotypes of all conditions were also evaluated (FIG. 3).

CAR T cell +BEAT and MoDCs group exhibited reduced CAR T cell exhaustion markers LAG-3, PD-1 and Tim-3 compared to tumour stimulated CAR T cells, even following multiple stimulations with MoDCs. Comparatively, the CAR T cell +tumour group treated with multiple stimulations of tumour on day 2 and 4 had increased activation (CD25, CD69) and exhaustion (LAG-3, PD-1 and Tim-3) phenotypes. Activation and exhaustion was not as prevalent in the 6 day proliferation assay with CAR+tumour group which was not subject to multiple tumour stimulation (FIG. 2).

Furthermore, restimulation of CAR T cells with BEAT and MoDCs did not change CAR T cell memory phenotypes (TSCM and TCM) compared to CAR T cells without the BEAT and MoDCs additional stimulation. CAR T cell phenotypes following tumour restimulation, however, demonstrated increased effector memory phenotype (TEM) and reduced TSCM compared to the CAR T cells without the additional tumour stimulation. TCM and TSCM CAR T cell phenotypes were also similar between CAR T cell with tumour stimulation (no restimulation) and CAR T cells with BEAT and MoDCs both with and without restimulation for CD4+ and CD8+ subsets, although TSCM phenotypes were increased in the CD8+ subsets following stimulation (and restimulation) with the BEAT and MoDCs (FIG. 3).

### Example 2—Bispecific BEAT Rescues Exhausted Tumour Pre-Treated CAR T Cells

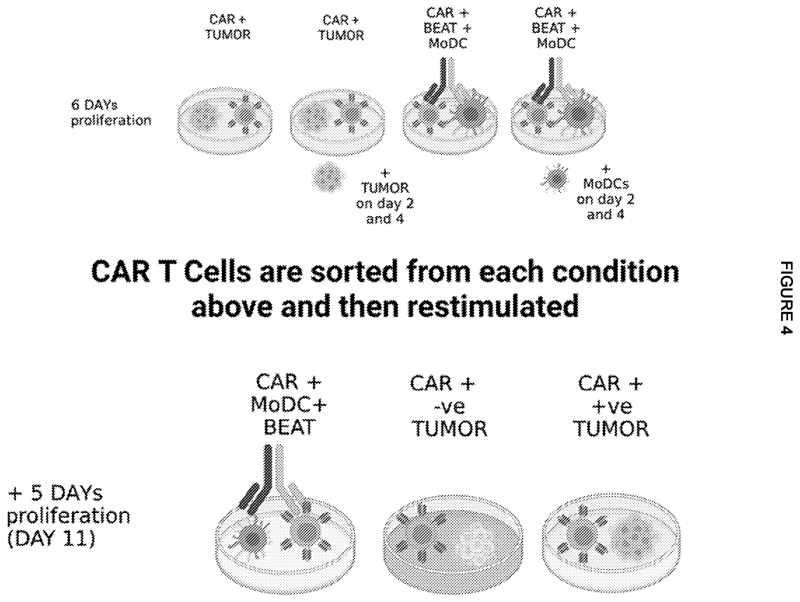

In this experiment, CAR T cells were sorted from each stimulation condition as described in the above Example and restimulated with either BEAT and MoDCs, mesothelin positive tumour or mesothelin negative tumour. (FIG. 4)

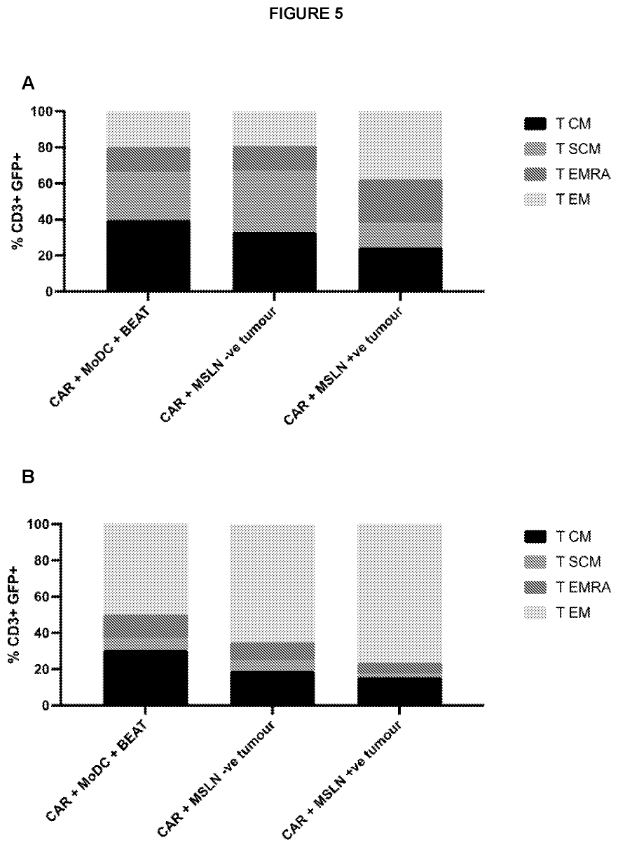

Phenotypes of all conditions were evaluated after a further 5 days of culturing (day 11) (FIG. 5). When CAR T cells were treated with mesothelin positive tumours, BEAT and MoDCs pre-treated CAR T cells showed higher central memory (TCM) and stem cell memory (TSCM) phenotypes than tumour pre-treated CAR T cells which have a higher effector memory phenotype (TEM). Surprisingly, re-stimulation of tumour pre-treated CAR T cells with BEAT and MoDCs rescued TCM phenotypes (FIG. 5B). Contrastingly, mesothelin positive tumour restimulation of the BEAT and MoDCs pre-treated CAR T cells had increased TCM and TSCM phenotypes (FIG. 5A) compared to CAR T cells that had been pre-treated with mesothelin positive tumour and restimulated with mesothelin positive tumour (FIG. 5B).

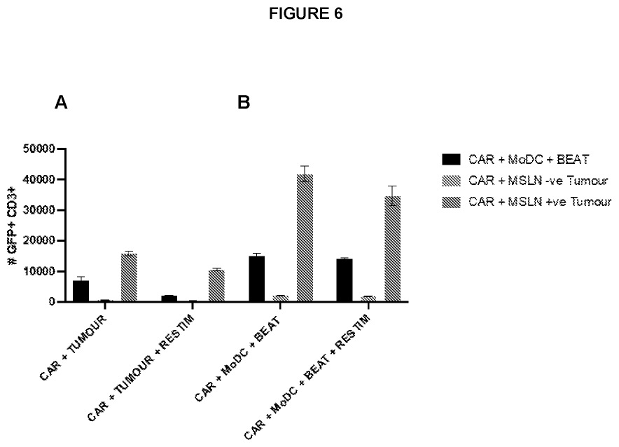

Furthermore, BEAT and MoDC pre-treated CAR T cells (FIG. 6B) demonstrated increased proliferation of CAR T cells compared to tumour pre-treated CAR T cells, which showed signs of exhaustion (FIG. 6A). Restimulation with either mesothelin positive tumour or MoDCs alone did not impact proliferation of the tumour pre-treated CAR T cells. Proliferation in the BEAT and MoDCs pre-treated CAR T cells was observed following restimulation with BEAT and MoDC or mesothelin positive tumour antigen, whereas no proliferation was seen in the presence of the mesothelin negative tumour (FIG. 6).

1. A method for rescuing immune effector cell activity or reversing immune effector cell exhaustion, the method comprising exposing an immune effector cell having or suspected of having an exhaustion phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cell,
optionally wherein the immune effector cell expresses a heterologous receptor comprising an antigen binding protein and a signalling protein,
thereby rescuing immune effector cell activity or reversing immune effector cell exhaustion.

2. A method for increasing immune effector cell activity or cytotoxicity of exhausted immune effector cells, the method comprising exposing a plurality of immune effector cells exhibiting one or more signs of an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cells,
optionally wherein the immune effector cells express heterologous receptors comprising an antigen binding protein and a signalling protein,
thereby increasing immune cell effector activity or cytotoxicity.

3. A method for preventing, inhibiting, reducing the progression of, or delaying the onset of, immune effector cell exhaustion, the method comprising exposing a plurality of immune effector cells having or suspected of having an exhausted phenotype, to an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on the immune effector cells,
optionally wherein the immune effector cells express heterologous receptors comprising an antigen binding protein and a signalling protein,
thereby preventing, inhibiting, reducing the progression of, or delaying the onset of, immune effector cell exhaustion.

4. A method for treating a disease or condition characterised by exhaustion of immune effector cells, the method comprising administering to a subject in need thereof, an effective amount of a bispecific polypeptide,
wherein the bispecific polypeptide comprises a first antigen binding protein that specifically binds to an antigen expressed on an antigen presenting cell (APC), preferably a professional APC, and a second binding antigen binding protein that specifically binds to an antigen on immune effector cells of the subject,
thereby treating a disease or condition characterised by exhaustion of immune effector cells.

5. The method of any one of claims 1 to 4, wherein the immune effector cell is a T cell, an NK cell, an NKT cell or a γδ T cell.

6. The method of any one of claims 1 to 5, wherein the effector immune cell is a T cell.

7. The method of any one of claims 1 to 6, wherein the immune effector cell expresses a chimeric antigen receptor (CAR) or an engineered T cell receptor (TCR) that binds to a target antigen (such as a tumour associated antigen).

8. The method of any one of claims 1 to 7, wherein the immune effector cell is a CAR T cell, and the bispecific polypeptide is for binding to an antigen on the CAR T cell.

9. The method of claim 1, wherein the immune effector cell is a CAR T cell, and the method is for rescuing CAR T cell activity or reversing CAR T cell exhaustion.

10. The method of claim 2, wherein the immune effector cell is a CAR T cell, and the method is for increasing CAR T cell activity or cytotoxicity of exhausted CAR T cells, preferably wherein the cells have or are suspected of having an exhausted phenotype.

11. The method of claim 3, wherein the immune effector cell is a CAR T cell, and wherein the method is for preventing, inhibiting, reducing the progression of, or delaying the onset of, CAR T cell exhaustion, preferably wherein the cells have or are suspected of having an exhausted phenotype.

12. The method of any one of claims 1 to 11, wherein the method comprises reversing at least one marker or sign of an exhaustion phenotype in an immune effector cell.

13. The method of any one of claims 1 to 12, wherein exposing the immune effector cells to the bispecific polypeptide comprises contacting a population of immune effector cells that have been obtained from a subject, with an effective amount of the bispecific polypeptide, ex vivo.

14. The method of claim 13, wherein the method comprises the step of further administering the immune effector cells that have been contacted with the bispecific polypeptide, to the subject.

15. The method of any one of claims 1 to 12, wherein exposing the immune cells to an effective amount of bispecific polypeptide comprises administration of the bispecific polypeptide to a subject in whom rescue or reverse of immune effector cell exhaustion, or increase of activity of exhausted immune effector cells, or preventing, inhibiting, reducing the progression of, or delaying the onset of immune effector cell exhaustion is required.

16. A method of:
rescuing immune effector cell (eg CAR T cell) activity;
reversing immune effector cell (eg CAR T cell) exhaustion;
increasing activity or cytotoxicity of exhausted immune effector cell (eg CAR T cells); or
preventing, inhibiting, reducing progression of, or delaying the onset of, immune effector cell (eg CAR T cell) exhaustion;
in a subject,
the method comprising administering to the subject, a bispecific polypeptide wherein the bispecific polypeptide comprises a first antigen binding protein that specifically binds to an antigen expressed on an antigen presenting cell (APC), preferably a professional APC, and a second binding antigen binding protein that specifically binds to an antigen on the immune (eg CAR T) cells.

17. The method of any one of claims 1 to 16, wherein the bispecific polypeptide is administered to a subject, after the subject has received a treatment with CAR T cells or other immune effector cell treatment.

18. The method of claim 17, wherein the bispecific polypeptide is administered to the subject at least 1 week, at least 2 weeks, at least 3 weeks, at least 4 weeks, at least 5 weeks or at least 6 weeks or longer following administration of the CAR T cells (or other immune effector cell treatment) to the subject.

19. The method of any one of claims 1 to 18, wherein at the time of administration of the bispecific polypeptide to a subject, an exhausted phenotype of one or more of the immune effector cells, or a marker or parameter indicative thereof, is detectable or measurable in the subject or in a biological sample from the subject.

20. The method of any one of claims 1 to 19, wherein at the time of administration of the bispecific polypeptide to a subject, an exhausted phenotype of one or more of the immune effector cells, or a marker or parameter indicative thereof, has been detected or measured in the subject or in a biological sample from the subject.

21. The method of any one of claims 1 to 19, wherein the method comprises determining the risk or likelihood that a subject has immune effector cells (such as CAR T cells) with an exhaustion phenotype.

22. The method of claim 20, wherein determining whether a subject has or is at risk of having immune effector cells (such as CAR T cells) with an exhausted phenotype comprises determining the expression of one or more genes associated with immune cell exhaustion, in a sample of immune effector cells obtained from the subject and/or determining an increase in the level or degree of surface expression of a marker on the immune cells, and/or determining the percentage of immune cells exhibiting surface expression, of one or more exhaustion markers, compared to a reference population of immune effector cells.

23. Use of a bispecific polypeptide in the manufacture of a medicament for:
rescuing or reversing immune effector cell (eg CAR T cell) exhaustion;
increasing immune effector cell activity or cytotoxicity of exhausted immune effector cells (eg CAR T cell); or
preventing, inhibiting, reducing progression of, or delaying the onset of, immune effector cell (eg CAR T cell) exhaustion
in a subject, preferably wherein the subject has received or is receiving a treatment with an engineered immune effector cell (such as a CAR T cell).

24. The method of any one of claims 1 to 23, wherein the methods are for treatment of a disease or condition selected from: cancer, a chronic infection (such as a chronic bacterial or chronic viral infection), chronic inflammation or autoimmunity.

25. The method of any one of claims 1 to 24, wherein the bispecific polypeptide binds to a professional APC that is an endogenous APC of the subject requiring treatment.

26. The method of any one of claims 1 to 25, wherein the bispecific polypeptide binds to an APC selected from a dendritic cell, a macrophage and a B-cell, more preferably, a dendritic cell (DC).

27. The method of any one of claims 1 to 26, wherein the bispecific polypeptide binds to an antigen of a professional APC selected from: MHCII, Clec9a, PD-L1, PD-L2, galectin, CD11c, CD19, CD40, CD206 and CD83.

28. The method of any one of claims 1 to 27, wherein the bispecific polypeptide binds to an antigen on the CAR portion of the CAR T cell.

29. The method of any one of claims 1 to 28, wherein the bispecific polypeptide binds to the antigen binding protein of the CAR, the hinge region of the CAR, a tag on the CAR (such as a Myc, Flag, His, HA, SBP, GST, MBP, GFP, S, Strep, exact or other tag) or any other region of the extracellular portion of the CAR.

30. The method of any one of claims 1 to 28, wherein the bispecific polypeptide binds to an antigen on the immune effector cell that is not an antigen on a heterologous receptor, such as a CAR.

31. The method of claim 30, wherein the bispecific polypeptide binds to an antigen on the surface of the immune effector cell selected from: CD3, CD4, CD8, CD25, CD127, CD196 (CCR6), CD27, CD28, CD45RA, CD45RO, CD62L, CD197, and HLA-DR).

32. The method of claim 30, wherein the bispecific polypeptide binds to a heterologous tag on the immune effector cell.

33. The method of any one of claims 1 to 32, wherein bispecific polypeptide is in the form of a fusion protein, optionally wherein the first and second antigen binding proteins are directly linked or linked via a hinge or linker region. Suitable linkers are further described herein.

34. The method of any one of claims 1 to 33, wherein the first and second antigen binding proteins of the bispecific polypeptide are in the form of an antibody, or antigen binding fragment thereof.

35. The method of claim 34, wherein the first antigen binding proteins is in the form of an antibody, such as an immunoglobulin G (IgG1, IgG2, IgG3, or IgG4).

36. The method of claim 35, wherein the first antigen binding protein is in the form of an antibody fragment (such as an scFv, a dimeric scFv (di-scFv), a Fab, a Fv, a F(ab′)2).

37. The method of claim 35 or 36, wherein the second antigen binding proteins is in the form of an antibody, such as an immunoglobulin G (IgG1, IgG2, IgG3, or IgG4).

38. The method of claim 35 or 36, wherein the second antigen binding proteins is in the form of an antibody fragment (such as an scFv, a dimeric scFv (di-scFv), a Fab, a Fv, a F(ab′)2).

39. The method of any one of claims 1 to 38, wherein the second antigen binding protein is linked to the first antigen binding protein via the C or N terminus, or an internal region of the first antigen binding protein, preferably wherein the second antigen binding protein is linked to the first antigen binding protein via the C terminus of the heavy chain of the first antigen binding protein.

40. The method of any one of claims 1 to 39, wherein the bispecific polypeptide comprises a first antigen binding protein for binding to CD40.

41. The method of any one of claims 1 to 40, wherein the bispecific polypeptide comprises a second antigen binding protein for binding to a tag on a CAR of a CAR T cell.

42. The method of claim 41, wherein the second antigen binding protein is for binding to a FLAG tag.

43. The method of any one of claims 1 to 42, wherein the bispecific polypeptide comprises a first antigen binding protein for binding to CD40, and wherein the first antigen binding protein comprises a CDRH1, a CDRH2 and/or a CDRH3 of an antigen binding domain having a variable heavy domain as defined in SEQ ID NO: 1 and/or a CDRL1, a CDRL2 and/or a CDRL3 of an antigen binding domain having a variable light chain as defined in of SEQ ID NO: 2.

44. The method of any one of claims 1 to 43, wherein the first antigen binding protein comprises:
FR1-CDR1-FR2-CDR2-FR3-CDR3-FR4-linker-FR1a-CDR1a-FR2a-CDR2a-FR3a-CDR3a-FR4a,
optionally, wherein the linker may be a chemical, one or more amino acids, or a disulphide bond formed between two cysteine residues; and
wherein the first antigen binding protein comprises an antigen binding domain comprising:
(i) a VH comprising a complementarity determining region (CDR) 1 comprising or consisting of a sequence as set forth in SEQ ID NO: 3, a CDR2 comprising or consisting of a sequence as set forth in SEQ ID NO: 4 and a CDR3 comprising or consisting of a sequence as set forth in SEQ ID NO: 5, and
(ii) a VL comprising a complementarity determining region (CDR) 1 comprising or consisting of a sequence as set forth in SEQ ID NO: 6, a CDR2 comprising or consisting of a sequence as set forth in SEQ ID NO: 7 and a CDR3 comprising or consisting of a sequence as set forth in SEQ ID NO: 8;
or
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO:
3, a CDR2 comprising or consisting of a sequence at least about 80%, at least 85%, at least 90%, at least 92%, at least 95%, at least 97%, at least 99% identical to a sequence set forth in SEQ ID NO: 4; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 5, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 6, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 7, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8.

45. The method of any one of claims 1 to 44, wherein the bispecific polypeptide comprises a first antigen binding protein comprising a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

46. The method of any one of claims 1 to 45, wherein the bispecific polypeptide comprises a first antigen binding protein that comprises a light chain variable domain comprising or consisting of the amino acid sequence as set forth in SEQ ID NO: 2; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

47. The method of any one of claims 1 to 46, wherein the bispecific polypeptide comprises a second antigen binding protein for binding to a FLAG tag, wherein the second antigen binding protein comprises a CDRH1, a CDRH2 and/or a CDRH3 of an antigen binding domain having a variable heavy chain as defined in SEQ ID NO: 15 and/or a CDRL1, a CDRL2 and/or a CDRL3 of an antigen binding domain having a variable light chain as defined in SEQ ID NO: 16.

48. The method of any one of claims 1 to 47, wherein the second antigen binding protein comprises:
FR1-CDR1-FR2-CDR2-FR3-CDR3-FR4-linker-FR1a-CDR1a-FR2a-CDR2a-FR3a-CDR3a-FR4a,
optionally wherein the linker may be a chemical, one or more amino acids, or a disulphide bond formed between two cysteine residues; and
wherein the second antigen binding domain comprises an antigen binding domain comprising:
(i) a VH comprising a complementarity determining region (CDR) 1 comprising or consisting of a sequence as set forth in SEQ ID NO: 23, a CDR2 comprising or consisting of a sequence as set forth in SEQ ID NO: 24 and a CDR3 comprising or consisting of a sequence as set forth in SEQ ID NO: 25, and
ii) a VL comprising a complementarity determining region (CDR) 1 comprising or consisting of a sequence as set forth in SEQ ID NO: 26, a CDR2 comprising or consisting of a sequence as set forth in SEQ ID NO: 27 and a CDR3 comprising or consisting of a sequence as set forth in SEQ ID NO: 28;
or
(iii) a VH comprising a complementarity determining region CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 23; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 24; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 25, and
(iv) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 26; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 27; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 22.

49. The method of any one of claims 1 to 48, wherein the bispecific polypeptide comprises a second antigen binding protein that comprises an antigen binding domain comprising a variable heavy chain comprising the amino acid sequence as set forth in SEQ ID NO: 15, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

50. The method of any one of claims 1 to 49, wherein the bispecific polypeptide comprises a second antigen binding protein that comprises an antigen binding domain comprising a variable light chain comprising the amino acid sequence as set forth in SEQ ID NO: 16; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto.

51. The method of any one of claims 1 to 50, wherein the bispecific polypeptide comprises, consists essentially of or consists of an amino acid sequence of SEQ ID NOS: 42 and 43.
